(19) United States
(12) Patent Application Publication
(10) Pub. No.: US 2026/0097107 A1
(43) Pub. Date: Apr. 9, 2026

(54) MENINGOCOCCUS VACCINES

(71) Applicant: GLAXOSMITHKLINE BIOLOGICALS SA, Rixensart (BE)

(72) Inventors: Alessia BIOLCHI, Siena (IT); Brunella BRUNELLI, Siena (IT); Marzia Monica GIULIANI, Siena (IT); Vega MASIGNANI, Siena (IT)

(73) Assignee: GLAXOSMITHKLINE BIOLOGICALS SA, Rixensart (BE)

(21) Appl. No.: 19/372,898

(22) Filed: Oct. 29, 2025

Related U.S. Application Data
(62) Division of application No. 18/329,701, filed on Jun. 6, 2023, now Pat. No. 12,485,164.
Application No. 18/329,701 is a division of application No. 15/319,665, now Pat. No. 11,707,513.

(30) Foreign Application Priority Data
Jul. 17, 2014 (EP) 14177563.5

(51) Int. Cl. A61K 39/095; A61K 39/00; A61K 39/116; C07K 14/22

(52) U.S. Cl. CPC A61K 39/095; A61K 39/00; A61K 39/116; C07K 14/22; A61K 2039/70; C07K 2319/00

(57) ABSTRACT

Meningococcal vaccines can be improved by including multiple alleles or variants of fHbp, in order to provide broader coverage of the diversity which is known for this protein, and/or by reducing the quantity of an OMV component in each dose.

### CROSS-REFERENCE TO RELATED APPLICATIONS

This Application is a Divisional of U.S. patent application Ser. No. 18/329,701, filed Jun. 6, 2023, (now U.S. Pat. No. 12,485,164 issued on Dec. 2, 2025), which is a Divisional Application of U.S. Ser. No. 15/319,665, filed Dec. 16, 2016, (now U.S. Pat. No. 11,707,513 issued on Jul. 25, 2023), which is the § 371 U.S. National Phase entry of Int'l App No. PCT/EP2015/066229 filed Jul. 16, 2015, which claims priority to Application No. EP 14177563.5, filed in the European Patent Office Jul. 17, 2014, all of which are incorporated herein by reference in their entireties.

### INCORPORATION-BY-REFERENCE OF SEQUENCE LISTING OR TABLE

The instant application contains a Sequence Listing which has been submitted electronically in XML file format and is incorporated herein by reference in its entirety. Said XML copy, created on Sep. 7, 2023, is named VN56308D2_US_XML_Seq_List.xml and is 73,728 bytes in size.

### TECHNICAL FIELD

This invention is in the field of meningococcal vaccination.

### BACKGROUND

Neisseria meningitidis is a Gram-negative encapsulated bacterium which colonises the upper respiratory tract of approximately 10% of human population. Conjugate vaccines are available against serogroups A, C, W135 and Y, but the only vaccine which is available for protecting against serogroup B in general is the BEXSERO™ product which was approved in 2013. This product includes four main immunogenic components: the factor H binding protein, ‘fHbp’: the heparin binding protein, NHBA: Neisserial adhesin A, NadA; and outer membrane vesicles (OMVs).

### SUMMARY OF THE INVENTION

An aspect of the present invention is an immunogenic composition comprising a fusion polypeptide comprising all three of v1, v2 and v3 meningococcal fHbp, in combination with one or more of (i) a NHBA polypeptide (ii) a NadA polypeptide and/or (iii) meningococcal outer membrane vesicles.

A further aspect of the invention is an immunogenic composition comprising meningococcal outer membrane vesicles in combination with one or more of (i) a NHBA polypeptide (ii) a NadA polypeptide and/or (iii) a fusion polypeptide comprising all three of v1, v2 and v3 meningococcal fHbp; where the outer membrane vesicles (OMVs) are present at a concentration between 5-30 μg/ml. Particularly the fusion polypeptide comprising all three of v1, v2 and v3 meningococcal fHbp is a stabilised and/or fHbp non-binding fusion polypeptide. Yet more particularly, the v1 fHbp comprises a mutation at position R41, for example an R41S mutation. Still yet more particularly, the v2 and v3 fHbp polypeptides comprise one or more stabilising and/or factor H (fH) non-binding mutations at the following positions numbered according to the full length sequences (SEQ ID NOs: 1 & 3) and also according to the ΔG sequences (SEQ ID NOs: 8 & 9):

| | | Stabilising | | fH non-binding |
|---|---|---|---|---|
| v2 | SEQ ID NO: 1 | Ser-58 | Leu-149 | Glu-266 |
| | SEQ ID NO: 8 | Ser-32 | Leu-123 | Glu-240 |
| v3 | SEQ ID NO: 3 | Ser-63 | Leu-157 | Glu-274 |
| | SEQ ID NO: 9 | Ser-32 | Leu-126 | Glu-243 |

A further aspect of the present invention is an immunogenic composition comprising a fusion polypeptide having an amino acid sequence of formula NH₂ -A-[-X-L]₃-B—COOH, where each X is a different variant fHbp sequence, L is an optional linker amino acid sequence, A is an optional N terminal amino acid sequence, and B is an optional C terminal amino acid sequence.

A further aspect of the present invention is a method for protecting a mammal, such as a human, against a meningococcal infection, comprising administering an immunogenic composition according to the invention.

### BRIEF DESCRIPTION OF THE DRAWINGS

FIG. 1 shows a RCD curve, with proportion on the y-axis (0.0 to 1.0) and SBA titer on the x-axis (0 to 256, in steps of 16). The top curve is group C: the group which reaches 0.0 soonest is S.

FIG. 2 provides a schematic of stabilising and factor H (fH) non-binding mutations introduced into the v1, v2 and v3 fHbp polypeptides to produce 731 S and 731 SNB fusion proteins.

FIG. 3(a)-(g) demonstrates that compositions comprising the 741-231 fusion (SEQ ID NO: 10) and 1/4OMV elicits higher GMTs than BEXSERO™ against seven strains tested (3a=v2, 3b=v2, 3c=v3, 3d=v3, 3e=v2, 3f=v2, 3g=v3).

### DETAILED DESCRIPTION

To enhance the BEXSERO™ product it would be advantageous to further enhance the coverage of BEXSERO™ against diverse meningococcal strains (in case of potential shifts and mutations as the vaccine's use spreads) and also to reduce the rare occurrences of fever which are sometimes seen when the vaccine is co-administered with routine infant vaccines [1]. With these aims the inventors have modified BEXSERO™ in two ways: (i) to include multiple alleles or variants of fHbp, in order to provide broader coverage of the diversity which is known for this protein; and (ii) to reduce the quantity of the OMV component in each dose. As shown herein, these two modifications indeed lead to an improvement in the vaccine.

Thus, in a first embodiment the invention provides an immunogenic composition comprising a fusion polypeptide comprising all three of v1, v2 and v3 meningococcal fHbp, in combination with one or more of (i) a NHBA polypeptide (ii) a NadA polypeptide and/or (iii) meningococcal outer membrane vesicles.

Furthermore, in a second embodiment the invention provides an immunogenic composition comprising meningococcal outer membrane vesicles in combination with one or more of (i) a NHBA polypeptide (ii) a NadA polypeptide and/or (iii) a fusion polypeptide comprising all three of v1, v2 and v3 meningococcal fHbp: wherein the outer membrane vesicles are present at a concentration between 5-30 μg/ml.

Similarly, combining both of these embodiments, the invention provides an immunogenic composition comprising a (i) a fusion polypeptide comprising all three of v1, v2 and v3 meningococcal fHbp. (ii) a NHBA polypeptide (iii) a NadA polypeptide and (iv) 5-30 μg/ml meningococcal outer membrane vesicles.

Factor H Binding Protein (fHbp)

A composition of the invention may include an immunogenic fHbp polypeptide. The BEXSERO™ product includes a fHbp polypeptide, and fHbp has also been known as ‘741’ (SEQ ID NO: 2536 in ref. 2: SEQ ID 1 herein), ‘NMB1870’, ‘GNA1870’ [3-5], ‘P2086’, ‘LP2086’ or ‘ORF2086’ [6-8]. The 3D structure of this protein is known [9,10], and the protein has two β-barrels connected by a short linker. Many publications have reported on the protective efficacy of this protein in meningococcal vaccines e.g. see references 11-15. This protein is expressed in lipidated form in multiple strains across all serogroups. fHbp sequences have been grouped into three variants [3] (referred to herein as v1, v2 and v3), and it has been found in general that serum raised against a given variant is bactericidal against strains which express that variant, but is not active against strains which express one of the other two variants i.e. there is intra-variant cross-protection, but not inter-variant cross-protection (except for some v2 and v3 cross-reactivity).

To increase inter-variant cross-reactivity the fHbp sequence has been engineered to contain specificities for all three variants [16]. Instead of following this approach, however, the invention utilises a fusion polypeptide which comprises all three of v1, v2 and v3 meningococcal fHbp.

v1 fHbp

Full-length fHbp from strain MC58 in v1 has the following amino acid sequence (SEQ ID NO: 1):

MNRTAFCCLSLTTALILTACSSGGGGVAADIGAGLADALTAPLDHKDKGLQSLTLDQSVRKNEKL
KLAAQGAEKTYGNGDSLNTGKLKNDKVSRFDFIRQIEVDGQLITLESGEFQVYKQSHSALTAFQT
EQIQDSEHSGKMVAKRQFRIGDIAGEHTSFDKLPEGGRATYRGTAFGSDDAGGKLTYTIDFAAKQ
GNGKIEHLKSPELNVDLAAADIKPDGKRHAVISGSVLYNQAEKGSYSLGIFGGKAQEVAGSAEVK
TVNGIRHIGLAAKQ

The mature lipoprotein lacks the first 19 amino acids of SEQ ID NO: 1 (underlined; provides SEQ ID NO: 4, beginning with Cys-20). The BEXSERO™ product includes a ‘ΔG’ form of v1 fHbp in which the full-length sequence is truncated up to residue 26 (i.e. to remove the poly-glycine stretch beginning instead with Val-27), giving SEQ ID NO: 7.

A v1 meningococcal fHbp used with the invention will comprise an amino acid sequence (i) with at least i % sequence identity to SEQ ID NO: 7, and/or (ii) comprising a fragment of SEQ ID NO: 7.

The value of i may be selected from 80, 85, 86, 87, 88, 89, 90, 91, 92, 93, 94, 95, 96, 97, 98, 99 or more. It is preferably 90 (i.e. the amino acid sequence has at least 90% identity to SEQ ID NO: 7) and is more preferably 95.

The fragment of (ii) will generally be at least 7 amino acids long e.g. 8, 10, 12, 14, 16, 18, 20, 22, 24, 26, 28, 30, 32, 24, 26, 28, 40, 45, 50, 55, 60, 65, 70, 75, 80 or more contiguous amino acids from SEQ ID NO: 7. The fragment will typically include at least one epitope from SEQ ID NO: 7. Epitope identification and mapping is established for fHbp [12:17-21]. Sharing at least 30 contiguous amino acids with SEQ ID NO: 7 will be typical, and usually a v1 fHbp amino acid sequence will include several (e.g. 2, 3, 4, 5 or more) fragments from SEQ ID NO: 7.

Overall, a v1 fHbp amino acid sequence can have at least i % sequence identity to and include several fragments of SEQ ID NO: 7.

A v1 fHbp sequence generally includes at least one amino acid sequence which is not present in SEQ ID NO: 2 and/or at least one amino acid sequence which is not present in SEQ ID NO: 3.

A polypeptide used with the invention and including a v1 sequence can, after administration to a suitable host mammal (such as a mouse or a human), elicit antibodies which can recognise a wild-type meningococcal polypeptide consisting of SEQ ID NO: 4. These antibodies will include some antibodies which do not recognise a v2 or a v3 polypeptide (e.g. will not recognise a wild-type meningococcal polypeptide consisting of SEQ ID NO: 5 and a wild-type meningococcal polypeptide consisting of SEQ ID NO: 6), although they may also include some antibodies which cross-react with v2 and/or v3 polypeptides. The antibodies are ideally bactericidal against a meningococcal strain which expresses a v1 fHbp e.g. against the MC58 strain (see below).

v2 fHbp

Full-length fHbp from strain 2996 in v2 has the following amino acid sequence (SEQ ID NO: 2):

MNRTAFCCLSLTAALILTACSSGGGGVAADIGAGLADALTAPLDHKDKSLQSLTLDQSVRKNEKL
KLAAQGAEKTYGNGDSLNTGKLKNDKVSRFDFIRQIEVDGQLITLESGEFQIYKQDHSAVVALQI
EKINNPDKIDSLINQRSFLVSGLGGEHTAFNQLPDGKAEYHGKAFSSDDAGGKLTYTIDFAAKQG
HGKIEHLKTPEQNVELAAAELKADEKSHAVILGDTRYGSEEKGTYHLALFGDRAQEIAGSATVKI
GEKVHEIGIAGKQ

The mature lipoprotein lacks the first 19 amino acids of SEQ ID NO: 2 (underlined: provides SEQ ID NO: 5), and the ΔG form of SEQ ID NO: 2 lacks the first 26 amino acids (SEQ ID NO: 8).

A v2 meningococcal fHbp used with the invention will comprise an amino acid sequence (i) with at least j % sequence identity to SEQ ID NO: 8, and/or (ii) comprising a fragment of SEQ ID NO: 8.

The value of j may be selected from 80, 85, 86, 87, 88, 89, 90, 91, 92, 93, 94, 95, 96, 97, 98, 99 or more. It is preferably 90 (i.e. the amino acid sequence has at least 90% identity to SEQ ID NO: 8) and is more preferably 95.

The fragment of (ii) will generally be at least 7 amino acids long e.g. 8, 10, 12, 14, 16, 18, 20, 22, 24, 26, 28, 30, 32, 24, 26, 28, 40, 45, 50, 55, 60, 65, 70, 75, 80 or more contiguous amino acids from SEQ ID NO: 8. The fragment will typically include at least one epitope from SEQ ID NO: 8. Epitope identification and mapping is established for fHbp (see above). Sharing at least 30 contiguous amino acids with SEQ ID NO: 8 will be typical, and usually a v2 fHbp amino acid sequence will include several (e.g. 2, 3, 4, 5 or more) fragments from SEQ ID NO: 8.

Overall, a v2 fHbp amino acid sequence can have at least j % sequence identity to and include several fragments of SEQ ID NO: 8.

A v2 fHbp sequence generally includes at least one amino acid sequence which is not present in SEQ ID NO: 1 and/or at least one amino acid sequence which is not present in SEQ ID NO: 3.

A polypeptide used with the invention and including a v2 sequence can, after administration to a suitable host mammal (such as a mouse or a human), elicit antibodies which can recognise a wild-type meningococcal polypeptide consisting of SEQ ID NO: 5. These antibodies will include some antibodies which do not recognise a v1 or a v3 polypeptide (e.g. will not recognise a wild-type meningococcal polypeptide consisting of SEQ ID NO: 4 and a wild-type meningococcal polypeptide consisting of SEQ ID NO: 6), although they may also include some antibodies which cross-react with v1 and/or v3 polypeptides. The antibodies are ideally bactericidal against a meningococcal strain which expresses a v2 fHbp e.g. against the M2091 strain (see below).

v3 fHbp

Full-length fHbp from strain M1239 in v3 has the following amino acid sequence (SEQ ID NO: 3):

MNRTAFCCLSLTTALILTACSSGGGGSGGGGVAADIGTGLADALTAPLDHKDKGLKSLTLEDSI
PQNGTLTLSAQGAEKTFKAGDKDNSLNTGKLKNDKISRFDFVQKIEVDGQTITLASGEFQIYKQN
HSAVVALQIEKINNPDKTDSLINQRSFLVSGLGGEHTAFNQLPGGKAEYHGKAFSSDDPNGRLHY
SIDFTKKQGYGRIEHLKTLEQNVELAAAELKADEKSHAVILGDTRYGSEEKGTYHLALFGDRAQE
IAGSATVKIGEKVHEIGIAGKQ

The mature lipoprotein lacks the first 19 amino acids of SEQ ID NO: 3 (underlined: provides SEQ ID NO: 6), and the ΔG form of SEQ ID NO: 3 lacks the first 31 amino acids (SEQ ID NO: 9).

A v3 meningococcal fHbp used with the invention will comprise an amino acid sequence (i) with at least k % sequence identity to SEQ ID NO: 9, and/or (ii) comprising a fragment of SEQ ID NO: 9.

The value of k may be selected from 80, 85, 86, 87, 88, 89, 90, 91, 92, 93, 94, 95, 96, 97, 98, 99 or more. It is preferably 90 (i.e. the amino acid sequence has at least 90% identity to SEQ ID NO: 9) and is more preferably 95.

The fragment of (ii) will generally be at least 7 amino acids long e.g. 8, 10, 12, 14, 16, 18, 20, 22, 24, 26, 28, 30, 32, 24, 26, 28, 40, 45, 50, 55, 60, 65, 70, 75, 80 or more contiguous amino acids from SEQ ID NO: 9. The fragment will typically include at least one epitope from SEQ ID NO: 9. Epitope identification and mapping is established for fHbp (see above). Sharing at least 30 contiguous amino acids with SEQ ID NO: 9 will be typical, and usually a v3 fHbp amino acid sequence will include several (e.g. 2, 3, 4, 5 or more) fragments from SEQ ID NO: 9.

Overall, a v3 fHbp amino acid sequence can have at least k % sequence identity to and include several fragments of SEQ ID NO: 9.

A v3 fHbp sequence generally includes at least one amino acid sequence which is not present in SEQ ID NO: 1 and/or at least one amino acid sequence which is not present in SEQ ID NO: 2.

A polypeptide used with the invention and including a v3 sequence can, after administration to a suitable host mammal (such as a mouse or a human), elicit antibodies which can recognise a wild-type meningococcal polypeptide consisting of SEQ ID NO: 6. These antibodies will include some antibodies which do not recognise a v1 or a v2 polypeptide (e.g. will not recognise a wild-type meningococcal polypeptide consisting of SEQ ID NO: 4 and a wild-type meningococcal polypeptide consisting of SEQ ID NO: 5), although they may also include some antibodies which cross-react with v1 and/or v2 polypeptides. The antibodies are ideally bactericidal against a meningococcal strain which expresses a v3 fHbp e.g. against the M01-240355 strain (see below).

### Fusion Polypeptide

The invention utilises a fusion polypeptide which comprises all three of v1, v2 and v3 meningococcal fHbp. As a result, the fusion polypeptide can include at least one epitope from each of SEQ ID NOs: 7, 8, and 9 and, after administration to a host mammal, can elicit antibodies which can recognise all three of (i) a wild-type meningococcal polypeptide consisting of SEQ ID NO: 4, (ii) a wild-type meningococcal polypeptide consisting of SEQ ID NO: 5, and (iii) a wild-type meningococcal polypeptide consisting of SEQ ID NO: 6. These antibodies are ideally bactericidal against a meningococcal strain which expresses a v1 fHbp, a meningococcal strain which expresses a v2 fHbp, and also a meningococcal strain which expresses a v3 fHbp (e.g. against each of the MC58, M2091, and M01-240355 strains).

With reference to the definitions given above, where relevant, for the fusion polypeptide it is preferred that i=j=k.

In general a fHbp fusion polypeptide of the invention has an amino acid sequence of formula:

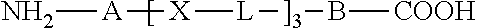

wherein each X is a different variant fHbp sequence, L is an optional linker amino acid sequence, A is an optional N-terminal amino acid sequence, and B is an optional C-terminal amino acid sequence.

The three X moieties are a v1, v2, and v3 sequence as discussed above. These can be present in any order from N- to C-terminus i.e. v1-v2-v3, v1-v3-v2, v2-v1-v3, v2-v3-v1, v3-v1-v2, or v3-v2-v1. The most preferred order is v2-v3-v1.

For each instance of [-X-L-], linker amino acid sequence -L- may be present or absent. Linker amino acid sequence(s) -L- will typically be short (e.g. 20 or fewer amino acids i.e. 19, 18, 17, 16, 15, 14, 13, 12, 11, 10, 9, 8, 7, 6, 5, 4, 3, 2, 1). Examples include short peptide sequences which facilitate cloning, poly-glycine linkers (i.e. Gly, where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. One useful linker is GSGGGG (SEQ ID NO: 22), with the Gly-Ser dipeptide being formed from a BamHI restriction site, thus aiding cloning and manipulation. Another useful linker is SEQ ID NO: 23, which can optionally be preceded by a Gly-Ser dipeptide (SEQ ID NO: 24, from BamHI) or a Gly-Lys dipeptide (SEQ ID NO: 25, from HindIII).

-A- is an optional N-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking. If X₁ lacks its own N-terminus methionine, -A- may provide such a methionine residue in the translated polypeptide (e.g. -A- is a single Met residue). The Met may be to the N-terminus of a linker sequence such as SEQ ID NO: 23 (i.e. SEQ ID: 26), or at the N-terminus of a short sequence (e.g. SEQ ID NO: 27).

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance polypeptide stability. Other suitable C-terminal amino acid sequences will be apparent to those skilled in the art. One suitable —B— moiety is SEQ ID NO: 28, in which the Leu-Glu upstream of the histidine tag arises from a XhoI restriction site.

One fusion polypeptide suitable for use with the invention comprises SEQ ID NO: 10. According to the above formula, in SEQ ID NO: 10 -A- is SEQ ID NO: 26, X₁ is a v2 fHbp sequence (SEQ ID NO: 8), -L₁- is SEQ ID NO: 24, X₂ is a v3 fHbp sequence (SEQ ID NO: 9), -L₂- is SEQ ID NO: 22, X₃ is a v1 fHbp sequence (SEQ ID NO: 7), and L₃ and B are absent. The three fHbp sequences in SEQ ID NO: 10 are underlined below:

MGPDSDRLQQRRVAADIGAGLADALTAPLDHKDKSLQSLTLDQSVRKNEKLKLAAQGAEKTYGNG
DSLNTGKLKNDKVSRFDFIRQIEVDGQLITLESGEFQIYKQDHSAVVALQIEKINNPDKIDSLIN
QRSFLVSGLGGEHTAFNQLPDGKAEYHGKAFSSDDAGGKLTYTIDFAAKQGHGKIEHLKTPEQNV
ELAAAELKADEKSHAVILGDTRYGSEEKGTYHLALFGDRAQEIAGSATVKIGEKVHEIGIAGKQG
SGPDSDRLQQRRVAADIGTGLADALTAPLDHKDKGLKSLTLEDSIPQNGTLTLSAQGAEKTFKAG
DKDNSLNTGKLKNDKISRFDFVQKIEVDGQTITLASGEFQIYKQNHSAVVALQIEKINNPDKTDS
LINQRSFLVSGLGGEHTAFNQLPGGKAEYHGKAFSSDDPNGRLHYSIDFTKKQGYGRIEHLKTLE
QNVELAAAELKADEKSHAVILGDTRYGSEEKGTYHLALFGDRAQEIAGSATVKIGEKVHEIGIAG
KQGSGGGGVAADIGAGLADALTAPLDHKDKGLQSLTLDQSVRKNEKLKLAAQGAEKTYGNGDSLN
TGKLKNDKVSRFDFIRQIEVDGQLITLESGEFQVYKQSHSALTAFQTEQIQDSEHSGKMVAKRQF
RIGDIAGEHTSFDKLPEGGRATYRGTAFGSDDAGGKLTYTIDFAAKQGNGKIEHLKSPELNVDLA
AADIKPDGKRHAVISGSVLYNQAEKGSYSLGIFGGKAQEVAGSAEVKTVNGIRHIGLAAKQ

A more preferred fusion polypeptide for use with the invention comprises SEQ ID NO: 29. According to the above formula, in SEQ ID NO: 29 -A- is SEQ ID NO: 26, X₁ is a v2 fHbp sequence (SEQ ID NO: 8), -L₁- is SEQ ID NO: 22, X₂ is a v3 fHbp sequence (SEQ ID NO: 9), -L₂- is SEQ ID NO: 22, X₃ is a v1 fHbp sequence (SEQ ID NO: 7), and L₃ and B are absent. The three fHbp sequences in SEQ ID NO: 29 are underlined below:

MGPDSDRLQQRRVAADIGAGLADALTAPLDHKDKSLQSLTLDQSVRKNEKLKLAAQGAEKTYGNG
DSLNTGKLKNDKVSRFDFIRQIEVDGQLITLESGEFQIYKQDHSAVVALQIEKINNPDKIDSLIN
QRSFLVSGLGGEHTAFNQLPDGKAEYHGKAFSSDDAGGKLTYTIDFAAKQGHGKIEHLKTPEQNV
ELAAAELKADEKSHAVILGDTRYGSEEKGTYHLALFGDRAQEIAGSATVKIGEKVHEIGIAGKQG
SGGGGVAADIGTGLADALTAPLDHKDKGLKSLTLEDSIPQNGTLTLSAQGAEKTFKAGDKDNSLN
TGKLKNDKISRFDFVQKIEVDGQTITLASGEFQIYKQNHSAVVALQIEKINNPDKTDSLINQRSF
LVSGLGGEHTAFNQLPGGKAEYHGKAFSSDDPNGRLHYSIDFTKKQGYGRIEHLKTLEQNVELAA
AELKADEKSHAVILGDTRYGSEEKGTYHLALFGDRAQEIAGSATVKIGEKVHEIGIAGKQGSGGG
GVAADIGAGLADALTAPLDHKDKGLQSLTLDQSVRKNEKLKLAAQGAEKTYGNGDSLNTGKLKND
KVSRFDFIRQIEVDGQLITLESGEFQVYKQSHSALTAFQTEQIQDSEHSGKMVAKRQFRIGDIAG
EHTSFDKLPEGGRATYRGTAFGSDDAGGKLTYTIDFAAKQGNGKIEHLKSPELNVDLAAADIKPD
GKRHAVISGSVLYNQAEKGSYSLGIFGGKAQEVAGSAEVKTVNGIRHIGLAAKQ

Thus the invention ideally utilises a polypeptide having amino acid sequence SEQ ID NO: or SEQ ID NO: 29, but the invention can also use a polypeptide comprising SEQ ID NO: 10 or SEQ ID NO: 29, but modified by up to 10 single amino acid changes (i.e. 1, 2, 3, 4, 5, 6, 7, 8, 9, or 10 single amino acid substitutions, deletions and/or insertions), provided that the polypeptide can elicit antibodies which can recognise all three of a wild-type meningococcal polypeptides of SEQ ID NOs: 4-6, as discussed above. Furthermore, SEQ ID NO: 10 or SEQ ID NO: 29 can be modified to change their -A- moiety (e.g. to use an alternative to SEQ ID NO: 26), so a polypeptide used with the invention can comprise SEQ ID NO: 30, optionally modified by up to 10 single amino acid changes (as discussed above).

For instance, SEQ ID NO: 30 can be modified to introduce point mutations which disrupt the ability of each fHbp to interact with fH. For example, SEQ ID NO: 30 can be mutated at residues E240, E496, and R543, thereby giving SEQ ID NO: 31 (comprising mutations E240X, E496X and R543X, where X is any amino acid other than the recited amino acid, i.e., E240X refers to any amino acid other than E at residue 240). A preferred embodiment of SEQ ID NO: 31 is SEQ ID NO: 32 (comprising the mutations E240A. E496A. R543S). The invention can use SEQ ID NO: 31 (e.g. SEQ ID NO: 32), optionally modified by up to 5 single amino acid changes (as discussed above), provided that residues E240, E496, and R543 are not present.

Furthermore. SEQ ID NO: 30 can be modified to introduce point mutations which increase the stability of a fHbp. For example, SEQ ID NO: 30 can be mutated at residues S32. L123, S285, and L379, thereby giving SEQ ID NO: 33 (comprising mutations S32X, L123X, S285X and L379X). A preferred embodiment of SEQ ID NO: 33 is SEQ ID NO: 34 (comprising mutations S32V, L123R, S285V, L379R). The invention can use SEQ ID NO: 33 (e.g. SEQ ID NO: 34), optionally modified by up to 5 single amino acid changes (as discussed above), provided that residues S32, L123, S285, and L379 are not present. One such polypeptide is SEQ ID NO: 35, in which the v1 sequence has been modified to include a mutation as reported in ref. 22 e.g. the ‘R41S’ mutation (SEQ ID NO: 36). SEQ ID NO: 35 comprises mutations S32X, L123X, S285X, L379X and R543X, and SEQ ID NO: 36 comprises mutations S32V, L123R, S285V, L379R and R543S. The ‘R41S’ nomenclature is numbered relative to the mature v1 polypeptide (SEQ ID NO:4), thus, e.g., it is present in the SEQ ID NO:35 fusion polypeptide as R543X and in SEQ ID NO:36 as R543S.

These various approaches can be combined, so the invention can utilise a polypeptide comprising SEQ ID NO: 37 (e.g. a polypeptide having amino acid sequence SEQ ID NO: 38). SEQ ID NO: 37 and SEQ ID NO: 38 comprise mutations S32V, L123R, E240A, S285V, L379R, E496A and R543S. SEQ ID NO:38 further comprises SEQ ID NO:26 at the N-terminal

In a further embodiment, the invention can use SEQ ID NO: 39 (comprising mutations L123X and L379X) e.g. SEQ ID NO: 40 (comprising mutations L123R and L379R). The invention can similarly use SEQ ID NO: 39 (e.g. SEQ ID NO: 40), optionally modified by up to 5 single amino acid changes (as discussed above), provided that residues L123 and L379 are not present (e.g. see SEQ ID NO: 34, which differs from SEQ ID NO: 40 by including two S/V substitutions as noted above). One such polypeptide is SEQ ID NO: 41, in which the v1 sequence has been modified to include the ‘R41S’ mutation, and thus comprises L123R, L379R and R543S. In further embodiments, when such fusion proteins are present in compositions of the invention. OMVs may be present at concentrations of between 2.5 μg/ml and 12.5 μg/ml.

The amino acid residues noted for mutation above are defined relative to specific starting sequences. The corresponding amino acid residues in any other fHbp sequence can be readily identified by sequence alignment e.g. being the amino acid which, when aligned using a pairwise alignment algorithm (e.g. the Needleman-Wunsch global alignment algorithm, as detailed below), aligns with the amino acid mentioned herein. Often the amino acid will be the same, but the alignment will easily identify if this is not the case.

The fHbp is naturally a lipoprotein in N. meningitidis. It has also been found to be lipidated when expressed in E. coli with its native leader sequence or with heterologous leader sequences. Polypeptides of the invention may have a N-terminus cysteine residue, which may be lipidated e.g. comprising a palmitoyl group, usually forming tripalmitoyl-S-glyceryl-cysteine. In usual embodiments, however, the fusion polypeptide of the invention is not lipidated (typically because the N-terminal -A- moiety does not direct lipidation) in the expression host.

### Neisserial Heparin Binding Antigen (NHBA)

A composition of the invention may include an immunogenic NHBA polypeptide. The NHBA antigen was included in the published genome sequence for meningococcal serogroup B strain MC58 as gene NMB2132 (GenBank accession number GI: 7227388: SEQ ID NO: 11 herein). The sequences of NHBA antigen from many strains have been published since then. For example, allelic forms of NHBA can be seen in FIGS. 5 and 15 of reference 24, and in example 13 and FIG. 21 of reference 2 (SEQ IDs 3179 to 3184 therein). Various immunogenic fragments of the NHBA antigen have also been reported, including the ΔG′ fragment of SEQ ID NO: 12. Preferred NHBA antigens for use with the invention comprise an amino acid sequence: (a) having 60% or more identity (e.g. 65%, 70%, 75%, 80%, 85%, 90%, 91%, 92%, 93%, 94%, 95%, 96%, 97%, 98%, 99%, 99.5% or more) to SEQ ID NO: 12; and/or (b) comprising a fragment of at least ‘n’ consecutive amino acids of SEQ ID NO: 12, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 12.

The most useful NHBA antigens of the invention can elicit antibodies which, after administration to a suitable host mammal (such as a mouse or a human), can bind to a meningococcal polypeptide consisting of amino acid sequence SEQ ID NO: 13.

Advantageous NHBA antigens for use with the invention can elicit bactericidal anti-meningococcal antibodies after administration to a mammalian subject.

A particularly preferred NHBA polypeptide for use with the invention comprises SEQ ID NO: 12, optionally modified by up to 3 single amino acid changes (i.e. 1, 2, or 3 single amino acid substitutions, deletions and/or insertions), provided that the polypeptide can elicit antibodies which can bind to SEQ ID NO: 13, as discussed above.

As seen in the BEXSERO™ product, the NHBA polypeptide can usefully be present as a fusion polypeptide e.g. fused to a NMB1030 polypeptide. In such fusion polypeptides NMB1030 is preferably downstream of NHBA. NMB1030 from strain MC58 has GenBank accession number GI: 7226269 (SEQ ID NO: 14 herein). A NMB1030 sequence for use with the invention can comprise an amino acid sequence: (a) having 60% or more identity (e.g. 65%, 70%, 75%, 80%, 85%, 90%, 91%, 92%, 93%, 94%, 95%, 96%, 97%, 98%, 99%, 99.5% or more) to SEQ ID NO: 14; and/or (b) comprising a fragment of at least ‘n’ consecutive amino acids of SEQ ID NO: 14, wherein ‘n’ is 30 or more. One useful NMB1030 fragment is SEQ ID NO: 15.

One such NHBA-NMB1030 fusion polypeptide has amino acid sequence SEQ ID NO: 16. Thus the invention can use SEQ ID NO: 16, optionally modified by up to 3 single amino acid changes (i.e. 1, 2, or 3 single amino acid substitutions, deletions and/or insertions), provided that the polypeptide can elicit antibodies which can bind to SEQ ID NO: 13, as discussed above.

### Neisserial Adhesin a (NadA)

A composition of the invention may include an immunogenic NadA polypeptide. The NadA antigen was included in the published genome sequence for meningococcal serogroup B strain MC58 as gene NMB1994 (GenBank accession number GI: 7227256; SEQ ID NO: 17 herein). The sequences of NadA antigen from many strains have been published since then, and the protein's activity as a Neisserial adhesin has been well documented. Various immunogenic fragments of NadA have also been reported. Preferred NadA antigens for use with the invention comprise an amino acid sequence: (a) having 60% or more identity (e.g. 65%, 70%, 75%, 80%, 85%, 90%, 91%, 92%, 93%, 94%, 95%, 96%, 97%, 98%, 99%, 99.5% or more) to SEQ ID NO: 17; and/or (b) comprising a fragment of at least ‘n’ consecutive amino acids of SEQ ID NO: 17, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 17. The most useful NadA antigens of the invention can elicit antibodies which, after administration to a host mammal, can bind to a meningococcal polypeptide consisting of amino acid sequence SEQ ID NO: 18. Advantageous NadA antigens for use with the invention can elicit bactericidal anti-meningococcal antibodies after administration to a host mammal.

A particularly preferred NadA polypeptide for use with the invention has SEQ ID NO: 19, optionally modified by up to 3 single amino acid changes (i.e. 1, 2, or 3 single amino acid substitutions, deletions and/or insertions), provided that the polypeptide can elicit antibodies which can bind to SEQ ID NO: 18, as discussed above.

### Meningococcal Outer Membrane Vesicles (OMVs)

Compositions of the invention include meningococcal OMVs i.e. any proteoliposomic vesicle obtained by disruption of or blebbing from a meningococcal outer membrane to form vesicles therefrom that retain protein components of the outer membrane (e.g. PorA, PorB, RmpM, Opa, Opc, Omp85, FetA/FrpB, NspA, etc.), having a diameter in the range of 50-200 nm. Thus the term can include OMVs (sometimes referred to as ‘blebs’) as well as the vesicles referred to as microvesicles (MVs [25]) or ‘native OMVs’ (NOMVs' [26]). See also references 27 to 33. Typical outer membrane vesicles are prepared artificially from bacteria, and may be prepared using detergent treatment (e.g. with deoxycholate), or by non-detergent means (e.g. see reference 37). Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc., with sodium deoxycholate [34 & 35] being preferred for treating Neisseria) at a pH sufficiently high not to precipitate the detergent [36]. Other techniques may be performed substantially in the absence of detergent [37.38] using techniques such as sonication, homogenisation, microfluidisation, cavitation, osmotic shock, grinding. French press, blending, etc. Methods using no or low detergent can retain useful antigens such as NspA and fHbp [37]. Thus OMVs used with the invention may be prepared using an OMV extraction buffer having about 0.5% deoxycholate or lower e.g. about 0.2%, about 0.1%, <0.05% or even zero.

The vesicles known as MVs and NOMVs are naturally-occurring membrane vesicles that form spontaneously during bacterial growth and are released into culture medium. MVs can be obtained by culturing Neisseria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 45 & 46 describe Neisseria with high MV production.

Vesicles may be prepared from bacteria which have been genetically manipulated [39-42] e.g. to increase immunogenicity (e.g. hyper-express immunogens), to reduce toxicity, to inhibit capsular polysaccharide synthesis, to down-regulate PorA expression, etc. They may be prepared from hyperblebbing strains [43-46]. Vesicles from bacteria with different class I outer membrane protein subtypes may be used e.g. six different subtypes [47.48] using two different genetically-engineered vesicle populations each displaying three subtypes, or nine different subtypes using three different genetically-engineered vesicle populations each displaying three subtypes, etc. Useful subtypes include: P1.7.16; P1.5-1,2-2: P1.19, 15-1: P1.5-2, 10: P1.12-1, 13: P1.7-2,4; P1.22, 14: P1.7-1, 1: P1.18-1,3,6. In general, however, it is preferred for the present invention to prepare OMVs from a wild-type meningococcus strain.

Vesicles for use with the invention can thus be prepared from any wild-type meningococcal strain. The vesicles will usually be from a serogroup B strain, but it is possible to prepare them from serogroups other than B (e.g. reference 36 discloses a process for serogroup A), such as A, C, W135 or Y. The strain may be of any serotype (e.g. 1, 2a, 2b, 4, 14, 15, 16, etc.), any serosubtype (e.g. P1.4), and any immunotype (e.g. L1: L2: L3: L3,7: L3,7,9; L10; etc.). The meningococci may be from any suitable lineage, including hyperinvasive and hypervirulent lineages e.g. any of the following seven hypervirulent lineages: subgroup I; subgroup III; subgroup IV-1; ET-5 complex; ET-37 complex; A4 cluster; lineage 3. Most preferably, OMVs are prepared from the strain NZ98/254, or another strain with the P1.4 PorA serosubtype. The invention advantageously uses the same OMVs which are used in the BEXSERO™ and MENZB™ products, prepared from the strain NZ98/254.

Vesicles will generally include meningococcal LOS (also known as LPS), but the pyrogenic effect of LOS in OMVs is much lower than seen with the same amount of purified LOS, and adsorption of OMVs to aluminium hydroxide further reduces pyrogenicity. LOS levels are expressed in International Units (IU) of endotoxin and can be tested by the LAL assay (limulus amebocyte lysate). Preferably, LOS is present at less than 2000 IU per μg of OMV protein.

When LOS is present in a vesicle it is possible to treat the vesicle so as to link its LOS and protein components (“intra-bleb” conjugation [49]).

A useful process for OMV purification is described in reference 50 and involves ultrafiltration on crude OMVs, rather than instead of high speed centrifugation. The process may involve a step of ultracentrifugation after the ultrafiltration takes place. OMVs can also be purified using the two stage size filtration process described in ref. 51.

OMVs can usefully be suspended in a sucrose solution after they have been prepared.

### Combinations

A composition of the invention can include each of (a) a fusion polypeptide comprising all three of v1, v2 and v3 meningococcal fHbp (b) a NHBA polypeptide (c) a NadA polypeptide and (d) OMVs.

In such combinations: (a) the fHbp fusion polypeptide ideally comprises amino acid sequence SEQ ID NO: 10, but optionally modified by up to 10 single amino acid changes, as discussed above: (b) the NHBA polypeptide ideally comprises amino acid sequence SEQ ID NO: 12, but optionally modified by up to 3 single amino acid changes, as discussed above; and (c) the NadA polypeptide ideally comprises amino acid sequence SEQ ID NO: 19, but optionally modified by up to 3 single amino acid changes, as discussed above.

More preferably: (a) the fHbp fusion polypeptide has amino acid sequence SEQ ID NO: 10; (b) the NHBA polypeptide comprises amino acid sequence SEQ ID NO: 12; and (c) the NadA polypeptide has amino acid sequence SEQ ID NO: 19.

Even more preferably: (a) the fHbp fusion polypeptide has amino acid sequence SEQ ID NO: 10; (b) the NHBA polypeptide has amino acid sequence SEQ ID NO: 16; and (c) the NadA polypeptide has amino acid sequence SEQ ID NO: 19.

The polypeptides in compositions of the invention can be present at any concentration which results in an effective immunological response in a host. This dosing can be established through routine testing, particularly in view of the guidance provided by the BEXSERO™ product, which has fHbp. NHBA and NadA polypeptides each present at 100 μg/ml. Thus fHbp, NHBA and/or NadA polypeptides may each be present in a composition of the invention at a concentration of between 20 μg/ml and 400 μg/ml e.g. between 50-150 μg/ml, between 80-120 μg/ml, or about 100 μg/ml. Antigen concentrations are easily quantified by standard protein assays.

Similarly, OMVs in compositions of the invention can be present at any concentration which results in an effective immunological response in a host. This dosing can be established through routine testing, particularly in view of the guidance provided by the BEXSERO™ product, in which OMVs are present at 50 μg/ml. Thus, according to the first embodiment of the invention, OMVs may be present in a composition at a concentration of between 20 μg/ml and 100 μg/ml e.g. between 30-75 μg/ml, between 40-60 μg/ml, or ideally about 50 μg/ml. In the second embodiment of the invention, however, OMVs are present at a lower concentration, namely between 5 μg/ml and 30 μg/ml e.g. between 10 μg/ml and 15 μg/ml, or ideally about 12.5 μg/ml. In certain embodiments, OMVs are present at lower concentrations of between 2.5 μg/ml and 12.5 μg/ml, for example at 2.5 μg/ml. 3.0 μg/ml. 3.5 μg/ml. 4.0 μg/ml, 4.5 μg/ml, 5.0 μg/ml, 5.5 μg/ml, 6.0 μg/ml, 6.5 μg/ml. 7.0 μg/ml, 7.5 μg/ml. 8.0 μg/ml, 8.5 μg/ml, 9.0 μg/ml, 9.5 μg/ml and 10 μg/ml.

OMV quantities and concentrations in compositions of the invention are defined in the same manner as in the BEXSERO™ product, namely by reference to their total protein content. This can be assessed using various assays e.g. ref. 29 discloses use of the Folin-Lowry assay. Total protein can be as saved according to the European Pharmacopoeia, Ph. Eur. Assay 2.5.33, using any of the seven pharmacopoeial methods. Method 2 provides the Lowry test, which is preferred. Thus a composition of the second embodiment of the invention includes OMVs with 5-30 μg/ml total protein.

### Polypeptides

Polypeptides of the invention can be prepared by various means e.g. by chemical synthesis (at least in part), by digesting longer polypeptides using proteases, by translation from RNA, by purification from cell culture (e.g. from recombinant expression or from N. meningitidis culture), etc. Heterologous expression in an E. coli host is a preferred expression route.

Polypeptides of the invention are ideally at least 100 amino acids long e.g. 150aa, 175aa, 200aa, 225aa, or longer. For instance, a fHbp fusion polypeptide will usually be at least 500aa long, a NHBA polypeptide will usually be at least 400aa long, and a NadA polypeptide will usually be at least 250aa long.

Polypeptides are preferably prepared in substantially pure or substantially isolated form (i.e. substantially free from other Neisserial or host cell polypeptides). In general, the polypeptides are provided in a non-naturally occurring environment e.g. they are separated from their naturally-occurring environment. In certain embodiments, the polypeptide is present in a composition that is enriched for the polypeptide as compared to a starting material. Thus purified polypeptide is provided, whereby purified means that the polypeptide is present in a composition that is substantially free of other expressed polypeptides, whereby substantially free is meant that more than 50% (e.g. >75%, ≥80%, ≥90%, ≥95%, or ≥99%) of total polypeptide in the composition is a polypeptide of the invention.

Polypeptides can take various forms (e.g. native, fusions, non-glycosylated, lipidated, disulfide bridges, etc.).

Sequences such as SEQ ID NO: 19 do not include a N-terminus methionine. If a polypeptide of the invention is produced by translation in a biological host then a start codon is required, which will provide a N-terminus methionine in most hosts. Thus a polypeptide of the invention will, at least at a nascent stage, include a methionine residue upstream of said SEQ ID NO sequence.

In some embodiments a polypeptide in a composition of the invention can include a N-terminal sequence upstream of (as appropriate) the fHbp, NHBA or NadA polypeptide sequence. In some embodiments the polypeptide has a single methionine at the N-terminus immediately followed by the relevant immunogen's amino acid sequence: in other embodiments a longer upstream sequence may be used. Such an upstream sequence may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. a histidine tag i.e. Hisₙ where n=4, 5, 6, 7, 8, 9, 10 or more).

A polypeptide of the invention may also include amino acids downstream of the final amino acid of the fHbp, NHBA or NadA (as appropriate) amino acid sequence. Such C-terminal extensions may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising a histidine tag i.e. Hisₙ where n=4, 5, 6, 7, 8, 9, 10 or more), or sequences which enhance polypeptide stability. Other suitable C-terminal amino acid sequences will be apparent to those skilled in the art.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention: for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains.

Polypeptides of the invention are preferably expressed recombinantly in a heterologous host (for example, in E. coli), then purified, and then combined and formulated with OMVs to give a composition of the invention.

In some embodiments, a polypeptide comprises an amino acid sequence as described above, except that up to 10 amino acids (i.e. 1, 2, 3, 4, 5, 6, 7, 8, 9 or 10) at the N-terminus and/or up to 10 amino acids (i.e. 1, 2, 3, 4, 5, 6, 7, 8, 9 or 10) at the C-terminus are deleted.

### Bactericidal Responses

As mentioned above, preferred polypeptides and compositions of the invention can elicit antibody responses that are bactericidal against meningococci. Bactericidal antibody responses are conveniently measured after immunisation of mice and are a standard indicator of vaccine efficacy (e.g. see end-note 14 of ref. 52: also ref. 53). Thus the antibodies will be bactericidal against a test strain in a suitable serum bactericidal assay (SBA).

A fusion fHbp polypeptide can preferably elicit an antibody response which is bactericidal against a meningococcal strain which expresses a v1 fHbp, a meningococcal strain which expresses a v2 fHbp, and also a meningococcal strain which expresses a v3 fHbp. A suitable v1 strain for a SBA test is MC58, which is widely available (e.g. ATCC BAA-335) and was the strain sequenced in reference 23. A suitable v2 strain for a SBA test is M2091 (ATCC 13091). A suitable v3 strain for a SBA test is M01-240355, which is a Neisseria MLST reference strains (id 19265 in ref. 54) that has been fully sequenced (see EMBL ID CP002422 [55])

Thus preferred fHbp fusion polypeptides can elicit antibodies in a mouse which are bactericidal against each of strains MC58, M2091, and M01-240355 in a serum bactericidal assay. For example, a composition of the invention can provide a serum bactericidal titer of ≥1:4 using the Goldschneider assay with human complement [56-58], and/or providing a serum bactericidal titer of ≥1:128 using baby rabbit complement.

### Immunisation

Polypeptides as discussed above may be used as the active ingredient(s) of immunogenic compositions, and so the invention provides an immunogenic composition (e.g. a vaccine) of the invention comprising polypeptides as discussed above.

The invention also provides a method for raising an antibody response in a mammal, such as a mouse or a human, comprising administering an immunogenic composition of the invention to the mammal. The antibody response is preferably a protective and/or bactericidal antibody response. The invention also provides compositions of the invention for use in such methods.

The invention also provides a method for protecting a mammal, such as a mouse or a human, against a Neisserial (e.g. meningococcal) infection, comprising administering to the mammal an immunogenic composition of the invention.

The invention provides compositions of the invention for use as medicaments (e.g. as immunogenic compositions or as vaccines). In one embodiment, it also provides the use of a fusion polypeptide comprising all three of v1, v2 and v3 meningococcal fHbp, and one or more of (i) a NHBA polypeptide (ii) a NadA polypeptide and/or (iii) meningococcal outer membrane vesicles, in the manufacture of a medicament for preventing Neisserial (e.g. meningococcal) infection in a mammal. In another embodiment, the invention provides the use of meningococcal outer membrane vesicles and one or more of (i) a NHBA polypeptide (ii) a NadA polypeptide and/or (iii) a fusion polypeptide comprising all three of v1, v2 and v3 meningococcal fHbp, in the manufacture of a medicament for preventing Neisserial (e.g. meningococcal) infection in a mammal, wherein the concentration of outer membrane vesicles in the medicament is between 5-30 μg/ml.

The mammal is preferably a human. The human may be an adult or, preferably, a child. Where the vaccine is for prophylactic use, the human is preferably a child (e.g. a toddler or infant); where the vaccine is for therapeutic use, the human is preferably an adult. A vaccine intended for children may also be administered to adults e.g. to assess safety, dosage, immunogenicity, etc.

The uses and methods are particularly useful for preventing/treating diseases including, but not limited to, meningitis (particularly bacterial, such as meningococcal, meningitis) and bacteremia. For instance, they are suitable for active immunisation of individuals against invasive meningococcal disease caused by N. meningitidis (for example in serogroup B).

Efficacy of therapeutic treatment can be tested by monitoring Neisserial infection after administration of the composition of the invention. Efficacy of prophylactic treatment can be tested by monitoring immune responses against fHbp. NHBA. NadA and PorA (as appropriate) after administration of the composition. Immunogenicity of compositions of the invention can be determined by administering them to test subjects (e.g. children 12-16 months age, or animal models) and then determining standard parameters including serum bactericidal antibodies (SBA) and ELISA titres (GMT). These immune responses will generally be determined around 4 weeks after administration of the composition, and compared to values determined before administration of the composition. A SBA increase of at least 4-fold or 8-fold is preferred. Where more than one dose of the composition is administered, more than one post-administration determination may be made.

Preferred compositions of the invention can confer an antibody titre in a patient that is superior to the criterion for seroprotection for each antigenic component for an acceptable percentage of human subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

The invention may be used to elicit systemic and/or mucosal immunity.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is about 0.5 ml (e.g. as seen in the BEXSERO™ product).

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined. For instance, the BEXSERO™ product is administered as two or three doses given note less than 1 month or not less than 2 months apart, depending on the subject (e.g. infants or others).

The immunogenic composition of the invention will generally include a pharmaceutically acceptable carrier, which can be any substance that does not itself induce the production of antibodies harmful to the patient receiving the composition, and which can be administered without undue toxicity. Pharmaceutically acceptable carriers can include liquids such as water, saline, glycerol and ethanol. Auxiliary substances, such as wetting or emulsifying agents. pH buffering substances, and the like, can also be present in such vehicles. A thorough discussion of suitable carriers is available in ref. 59. For example, the BEXSERO™ product includes sodium chloride, histidine, sucrose, aluminium hydroxide, and water for injections.

Neisserial infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared. Compositions suitable for parenteral injection (e.g. to the muscle) are most preferred.

The composition is preferably sterile. It is preferably pyrogen-free. It is preferably buffered e.g. at between pH 6 and pH 8, generally around pH 7. Where a composition comprises an aluminium hydroxide salt, it is preferred to use a histidine buffer [60]. Compositions of the invention may be isotonic with respect to humans.

Immunogenic compositions comprise an immunologically effective amount of immunogen, as well as any other of other specified components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Dosage treatment may be a single dose schedule or a multiple dose schedule (e.g. including booster doses). The composition may be administered in conjunction with other immunoregulatory agents.

Adjuvants which may be used in compositions of the invention include, but are not limited to insoluble metal salts, oil-in-water emulsions (e.g. MF59 or AS03, both containing squalene), saponins, non-toxic derivatives of LPS (such as monophosphoryl lipid A or 3-O-deacylated MPL), immunostimulatory oligonucleotides, detoxified bacterial ADP-ribosylating toxins, microparticles, liposomes, imidazoquinolones, or mixtures thereof. Other substances that act as immunostimulating agents are disclosed in chapter 7 of ref. 61.

The use of an aluminium hydroxide and/or aluminium phosphate adjuvant is particularly preferred, and polypeptides are generally adsorbed to these salts. These salts include oxy hydroxides and hydroxyphosphates (e.g. see chapters 8 & 9 of ref. 61). The salts can take any suitable form (e.g. gel, crystalline, amorphous, etc.). Al⁺⁺⁺ should be present at <1 mg/dose. The most preferred adjuvant is aluminium hydroxide, as used in the BEXSERO™ product.

Polypeptides and OMVs in a composition of the invention can be adsorbed to this adjuvant, as seen in the BEXSERO™ product. Aluminium hydroxide can be included at about 1 mg/ml Al⁺⁺⁺ (i.e. 0.5 mg per 0.5 ml dose)

### Further Antigenic Components

A composition of the invention can include further meningococcal polypeptide immunogens in addition to fHbp, NHBA, NadA and/or OMVs. For instance, it might include one or more of NspA, App, NhhA, HmbR, etc.

A composition of the invention can also include a ‘936’ antigen. The 936 antigen was included in the published genome sequence for meningococcal serogroup B strain MC58 as gene NMB2091 (SEQ ID NO: 20 herein). Preferred 936 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 21; and/or (b) comprising a fragment of at least ‘n’ consecutive amino acids of SEQ ID NO: 21, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 21. The most useful 936 antigens of the invention can elicit antibodies which, after administration to a host mammal, can bind to a meningococcal polypeptide consisting of amino acid sequence SEQ ID NO: 20. The 936 antigen is a good fusion partner for fHbp (e.g. see references 62 & 63).

In addition to meningococcal polypeptide antigens, the composition may include antigens for immunising against other diseases or infections. For example, the composition may include one or more of the following further antigens:
a saccharide antigen from N. meningitidis serogroup A, C, W135 and/or Y, such as the saccharide disclosed in ref. 64 from serogroup C (see also ref. 65) or in ref. 66.
a saccharide antigen from Streptococcus pneumoniae [e.g. 67, 68, 69].
an antigen from hepatitis A virus, such as inactivated virus [e.g. 70, 71].
an antigen from hepatitis B virus, such as the surface and/or core antigens [e.g. 71, 72].
a diphtheria antigen, such as a diphtheria toxoid [e.g. chapter 3 of ref. 73] e.g. the CRM₁₉₇ mutant [e.g. 74].
a tetanus antigen, such as a tetanus toxoid (e.g. chapter 4 of ref. 73).
an antigen from Bordetella pertussis, such as pertussis holotoxin (PT) and filamentous haemagglutinin (FHA) from B. pertussis, optionally also in combination with pertactin and/or agglutinogens 2 and 3 (e.g. refs. 75 & 76).
a saccharide antigen from Haemophilus influenzae B [e.g. 65].
polio antigen(s) [e.g. 77, 78] such as IPV.
measles, mumps and/or rubella antigens (e.g. chapters 9, 10 & 11 of ref. 73).
influenza antigen(s) (e.g. chapter 19 of ref. 73), such as the haemagglutinin and/or neuraminidase surface proteins.
an antigen from Moraxella catarrhalis [e.g. 79].
an protein antigen from Streptococcus agalactiae (group B streptococcus) [e.g. 80, 81]
a saccharide antigen from Streptococcus agalactiae (group B streptococcus).
an antigen from Streptococcus pyogenes (group A streptococcus) [e.g. 81, 82, 83].
an antigen from Staphylococcus aureus [e.g. 84].

The composition may comprise one or more of these further antigens.

Toxic protein antigens may be detoxified where necessary (e.g. detoxification of pertussis toxin by chemical and/or genetic means [76]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. Carrier proteins for the conjugates are discussed in more detail below.

Antigens in the composition will typically be present at a concentration of at least 1 μg/ml each. In general, the concentration of any given antigen will be sufficient to elicit an immune response against that antigen.

Immunogenic compositions of the invention may be used therapeutically (i.e. to treat an existing infection) or prophylactically (i.e. to prevent future infection).

As an alternative to using proteins antigens in the immunogenic compositions of the invention, nucleic acid (which could be RNA, such as a self-replicating RNA, or DNA, such as a plasmid) encoding the antigen may be used.

In some embodiments a composition of the invention comprises conjugated capsular saccharide antigens from 1, 2, 3 or 4 of meningococcus serogroups A, C, W135 and Y. In other embodiments a composition of the invention comprises at least one conjugated pneumococcal capsular saccharide antigen.

### Meningococcus Serogroups Y, W135, C and A

Current serogroup C vaccines (MENJUGATE™ [64,85], MENINGITEC™ and NEISVAC-C™) include conjugated saccharides. MENJUGATE™ and MeningitecMENINGITEC™ have oligosaccharide antigens conjugated to a CRM₁₉₇ carrier, whereas NEISVAC-C™ uses the complete polysaccharide (de-O-acetylated) conjugated to a tetanus toxoid carrier. The MENACTRA™ vaccine contains conjugated capsular saccharide antigens from each of serogroups Y, W135, C and A.

Compositions of the present invention may include capsular saccharide antigens from one or more of meningococcus serogroups Y, W135, C and A, wherein the antigens are conjugated to carrier protein(s) and/or are oligosaccharides. For example, the composition may include a capsular saccharide antigen from: serogroup C: serogroups A and C: serogroups A, C and W135: serogroups A, C and Y: serogroups C, W135 and Y: or from all four of serogroups A, C, W135 and Y.

A typical quantity of each meningococcal saccharide antigen per dose is between 1 μg and 20 μg e.g. about 1 μg, about 2.5 μg, about 4 μg, about 5 μg, or about 10 μg (expressed as saccharide).

Where a mixture comprises capsular saccharides from both serogroups A and C, the ratio (w/w) of MenA saccharide:MenC saccharide may be greater than 1 (e.g. 2:1, 3:1, 4:1, 5:1, 10:1 or higher). Where a mixture comprises capsular saccharides from serogroup Y and one or both of serogroups C and W135, the ratio (w/w) of MenY saccharide:MenW135 saccharide may be greater than 1 (e.g. 2:1, 3:1, 4:1, 5:1, 10:1 or higher) and/or that the ratio (w/w) of MenY saccharide:MenC saccharide may be less than 1 (e.g. 1:2, 1:3, 1:4, 1:5, or lower). Preferred ratios (w/w) for saccharides from serogroups A:C:W135:Y are: 1:1:1:1; 1:1:1:2; 2:1:1:1; 4:2:1:1; 8:4:2:1; 4:2:1:2; 8:4:1:2; 4:2:2:1; 2:2:1:1; 4:4:2:1; 2:2:1:2; 4:4:1:2; and 2:2:2:1. Preferred ratios (w/w) for saccharides from serogroups C:W135:Y are: 1:1:1; 1:1:2; 1:1:1; 2:1:1; 4:2:1; 2:1:2; 4:1:2; 2:2:1; and 2:1:1. Using a substantially equal mass of each saccharide is preferred.

Capsular saccharides may be used in the form of oligosaccharides. These are conveniently formed by fragmentation of purified capsular polysaccharide (e.g. by hydrolysis), which will usually be followed by purification of the fragments of the desired size.

Fragmentation of polysaccharides is preferably performed to give a final average degree of polymerisation (DP) in the oligosaccharide of less than 30 (e.g. between 10 and 20, preferably around 10 for serogroup A: between 15 and 25 for serogroups W135 and Y, preferably around 15-20: between 12 and 22 for serogroup C: etc.). DP can conveniently be measured by ion exchange chromatography or by colorimetric assays [86].

If hydrolysis is performed, the hydrolysate will generally be sized in order to remove short-length oligosaccharides [65]. This can be achieved in various ways, such as ultrafiltration followed by ion-exchange chromatography. Oligosaccharides with a degree of polymerisation of less than or equal to about 6 are preferably removed for serogroup A, and those less than around 4 are preferably removed for serogroups W135 and Y.

Preferred MenC saccharide antigens are disclosed in reference 85, as used in MENJUGATE™

### Covalent Conjugation

Capsular saccharides in compositions of the invention will usually be conjugated to carrier protein(s). In general, conjugation enhances the immunogenicity of saccharides as it converts them from T-independent antigens to T-dependent antigens, thus allowing priming for immunological memory. Conjugation is particularly useful for paediatric vaccines and is a well known technique.

Typical carrier proteins are bacterial toxins, such as diphtheria or tetanus toxins, or toxoids or mutants thereof. The CRM₁₉₇ diphtheria toxin mutant is useful, and is the carrier in the PREVNAR™ product. Other suitable carrier proteins include the N. meningitidis outer membrane protein complex [88], synthetic peptides [89,90], heat shock proteins [91,92], pertussis proteins [93,94], cytokines [95], lymphokines [95], hormones [95], growth factors [95], artificial proteins comprising multiple human CD4⁺ T cell epitopes from various pathogen-derived antigens such as N19 [97], protein D from H.influenzae [98-100], pneumolysin or its non-toxic derivatives [102], pneumococcal surface protein PspA [103], iron-uptake proteins [104], toxin A or B from C. difficile [105], recombinant P. aeruginosa exoprotein A (rEPA) [106], etc.

Any suitable conjugation reaction can be used, with any suitable linker where necessary.

The saccharide will typically be activated or functionalised prior to conjugation. Activation may involve, for example, cyanylating reagents such as CDAP (e.g. 1-cyano-4-dimethylamino pyridinium tetrafluoroborate [107, 108, etc.]). Other suitable techniques use carbodiimides, hydrazides, active esters, norborane, p-nitrobenzoic acid, N-hydroxysuccinimide, S—NHS, EDC, TSTU, etc.

Linkages via a linker group may be made using any known procedure, for example, the procedures described in references 109 and 110. One type of linkage involves reductive amination of the polysaccharide, coupling the resulting amino group with one end of an adipic acid linker group, and then coupling a protein to the other end of the adipic acid linker group [111,112]. Other linkers include B-propionamido [113], nitrophenyl-ethylamine [114], haloacyl halides [115], glycosidic linkages [116], 6-aminocaproic acid [117], ADH [118], C₄ to C₁₂ moieties etc. As an alternative to using a linker, direct linkage can be used. Direct linkages to the protein may comprise oxidation of the polysaccharide followed by reductive amination with the protein, as described in, for example, references 120 and 121.

A process involving the introduction of amino groups into the saccharide (e.g. by replacing terminal ═O groups with —NH₂) followed by derivatisation with an adipic diester (e.g. adipic acid N-hydroxysuccinimido diester) and reaction with carrier protein is preferred. Another preferred reaction uses CDAP activation with a protein D carrier e.g. for MenA or MenC.

### General

The term “comprising” encompasses “including” as well as “consisting” e.g. a composition “comprising” X may consist exclusively of X or may include something additional e.g. X+Y. References to “comprising” (or “comprises”, etc.) may optionally be replaced by references to “consisting of” (or “consists of”, etc.).

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

“Sequence identity” is preferably determined by the Needleman-Wunsch global alignment algorithm [122], using default parameters (e.g. with Gap opening penalty=10.0, and with Gap extension penalty=0.5, using the EBLOSUM62 scoring matrix). This algorithm is conveniently implemented in the needle tool in the EMBOSS package [123]. Where the application refers to sequence identity to a particular SEQ ID, the identity should be calculated over the entire length of that SEQ ID.

After serogroup, meningococcal classification includes serotype, serosubtype and then immunotype, and the standard nomenclature lists serogroup, serotype, serosubtype, and immunotype, each separated by a colon e.g. B: 4:P1.15: L3,7,9. Within serogroup B, some lineages cause disease often (hyperinvasive), some lineages cause more severe forms of disease than others (hypervirulent), and others rarely cause disease at all. Seven hypervirulent lineages are recognised, namely subgroups I, III and IV-1, ET-5 complex, ET-37 complex, A4 cluster and lineage 3. These have been defined by multilocus enzyme electrophoresis (MLEE), but multilocus sequence typing (MLST) has also been used to classify meningococci. The four main hypervirulent clusters are ST32, ST44, ST8 and ST11 complexes.

### EXAMPLES

### Example 1: The BEXSERO™ Vaccine (for Reference)

The BEXSERO™ product is safe and effective and has been authorised for human use in Europe and elsewhere. It has the following immunogenic ingredients per 0.5 ml dose:

| Immunogen | Quantity | Notes |
|---|---|---|
| fHbp | 50 μg | Fusion polypeptide with NMB2091 |
| | | at N-terminus |
| NHBA | 50 μg | Fusion polypeptide with NMB1030 |
| | | at C-terminus |
| NadA | 50 μg | — |
| OMV | 25 μg | Strain NZ98/254 |
| | (total | (B:4:P1.7-2, 4, L1, 3) |
| | protein) | |

These immunogens are adsorbed to an aluminium hydroxide adjuvant (0.5 mg Al⁺⁺⁺ per dose). The composition also includes NaCl, a histidine buffer, and sucrose.

### Example 2: Stabilised and Stabilised Non-Binding Fusion Polypeptides

The inventors have studied two different types of mutation in v2 and v3: firstly, they have identified residues within SEQ ID NO: 2 and SEQ ID NO: 3 which can be modified to increase the polypeptide's stability. Secondly, they have identified residues which decrease binding to human factor H (fH). Mutant fHbp polypeptides comprising both types of mutation, have enhanced properties. Specifically, fHbp mutants that do not bind factor H but which retain immunogenicity are advantageous because the resultant antibody responses are directed towards epitopes in or near the fH-binding site. Following vaccination using wild-type fHbp vaccine antigens, such epitopes may be obscured by factor H binding. The amino acids of most interest are as follows, numbered according to the full-length sequences (SEQ ID NOs: 1 & 3) and also according to the ΔG sequences (SEQ ID NOs: 8 & 9):

| | | Stability** | | fH binding |
|---|---|---|---|---|
| v2 | SEQ ID NO: 1 | Ser-58 | Leu-149 | Glu-266 |
| | SEQ ID NO: 8 | Ser-32 | Leu-123 | Glu-240 |
| v3 | SEQ ID NO: 3 | Ser-63 | Leu-157 | Glu-274 |
| | SEQ ID NO: 9 | Ser-32 | Leu-126 | Glu-243 |

**Where only one of these residues is mutated, it is preferably the leucine

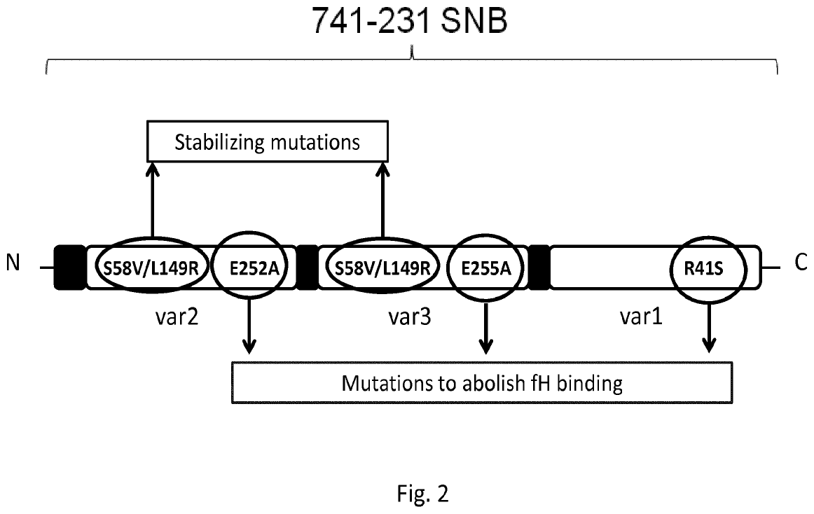

The mutations for stability and fHbp binding were combined into mutant forms of v2 and v3 and fused with a mutant v1 sequence comprising the R41S mutation. Mutants were fused in the order v2-v3-v1 and were joined using linkers, to give 731 SNB (SEQ ID NO: 38). Compared to the three wild-type sequences, this fusion polypeptide includes a total of 7 point mutations (FIG. 2).

Separately, the mutations for stability in v2 and v3 were fused with the ‘R41S’ mutant v1 sequence in the order v2-v3-v1 and were joined using linkers, to give 731 S (SEQ ID NO: 40). Thus, compared to the three wild-type sequences, this fusion polypeptide includes a total of 5 point mutations (FIG. 2).

The ability of non-fH binding forms of fHbp to elicits SBA titers was tested in transgenic (Tg) mice:

| | rSBA titers obtained against prototypic strains | | |
|---|---|---|---|
| Antigen | Var 1.1 | Var 2.16 | Var 3.42 |
| fHbp fusion | 1024* | 4096 | 8192 |
| SEQ ID NO: 10 | | | |
| fHbp fusion | 16384 | 32768 | >32768 |
| SEQ ID NO: 38 | | | |

These data indicate that non-binding forms of fHbp may be more immunogenic.

### Example 3: Substitution of NMB2091-fHbp Fusion

The BEXSERO™ product was modified by replacing the NMB2091-fHbp fusion polypeptide with a “triple fusion” polypeptide of fHbp variants, with v2-v3-v1 from N- to C-terminus. This fusion polypeptide has the amino acid sequence SEQ ID NO: 10. In addition, the OMV component was removed. The two vaccines were compared in mice immunised at days 0, 21 and 35, with sera being assessed at days 34 and 49 against a panel of 15 serogroup B strains in various clonal complexes, MLST, and ET classifications. Antigens were administered at 20 μg/dose, using the adjuvant at 3 mg/ml.

The proportion of strains with SBA titers above various thresholds were as follows:

| Threshold | Original vaccine | Modified vaccine |
|---|---|---|
| ≥128 | 100% | 100% |
| ≥1024 | 93% | 80% |
| >4096 | 53% | 60% |

Use of the v2-v3-v1 fusion polypeptide can thus provide cover against a higher proportion of the panel (60% vs. 53%) at a high anti-MenB SBA titer (>4096).

### Example 4:4-Fold Reduction of OMV Dosage

The BEXSERO™ product was modified by replacing the NMB2091-fHbp fusion polypeptide with the “triple fusion” fHbp v2-v3-v1 polypeptide (SEQ ID NO:10) but also by either (i) reducing the OMV dosage 4-fold to 12.5 μg/ml or (ii) removing the OMV component. Thus three compositions were prepared:

| Group | Protein immunogens | | | OMVs |
|---|---|---|---|---|
| M | NMB2091-fHbp | NHBA-NMB1030 | NadA | 50 μg/ml |
| C | fHbp-v2-v3-v1 | NHBA-NMB1030 | NadA | 12.5 μg/ml |
| S | fHbp-v2-v3-v1 | NHBA-NMB1030 | NadA | — |

To assess immunogenicity of these three vaccines human subjects received three doses at monthly intervals (months 0,1,2). Sera were taken at months 0, 1, 2 and 3, and then 6 months after the third dose (month 8), for assessment against a panel of relevant strains. Titers (GMT) were as follows:

| | | |
|---|---|---|
| M | C | S |

Strain H44/76

| | | | |
|---|---|---|---|
| Time zero | 1.36 | 2.16 | 1.55 |
| 1 month | 30 | 52 | 15 |
| 2 months | 97 | 91 | 48 |
| 3 months | 102 | 99 | 59 |
| 8 months | 25 | 33 | 12 |

Strain 5/99

| | | | |
|---|---|---|---|
| Time zero | 2.47 | 3.01 | 2.17 |
| 1 month | 70 | 75 | 56 |
| 2 months | 173 | 140 | 157 |
| 3 months | 237 | 236 | 365 |
| 8 months | 77 | 83 | 106 |

Strain NZ98/254

| | | | |
|---|---|---|---|
| Time zero | 1.21 | 2.04 | 1.73 |
| 1 month | 9.45 | 29 | 3.19 |
| 2 months | 13 | 12 | 4.4 |
| 3 months | 16 | 24 | 6.49 |
| 8 months | 3.55 | 8.02 | 3.55 |

Strain M14459

| | | | |
|---|---|---|---|
| Time zero | 1.86 | 2.48 | 2.16 |
| 2 months | 30 | 24 | 16 |
| 3 months | 34 | 31 | 19 |

Strain UK364

| | | | |
|---|---|---|---|
| Time zero | 1.35 | 1.97 | 2.07 |
| 2 months | 37 | 72 | 70 |
| 3 months | 56 | 113 | 112 |

Pooled patient sera were used to assess coverage of a panel of 7 MenB strains which express a v1 fHbp. A similar number of strains was adequately covered in each group, but titers (GMT) were highest in group C:

| | M | C | S |
|---|---|---|---|
| Time zero | <10 | <10 | <10 |
| 3 months | 70 | 140 | 40 |
| 8 months | 15 | 50 | 10 |

Single patient sera were tested against a panel of 6 MenB strains which express a v2 or v3 fHbp (one strain was tested twice). Again, titers (GMT) were highest in group C:

| | | |
|---|---|---|
| M | C | S |

Strain M14549 (v2)

| | | | |
|---|---|---|---|
| Time zero | 1.4 | 1.5 | 1.1 |
| 2 months | 3.8 | 15.0 | 6.2 |
| 3 months | 3.6 | 21.4 | 6.6 |

Strain M12566 (v2)

| | | | |
|---|---|---|---|
| Time zero | 6.0 | 10.7 | 14.8 |
| 2 months | 40.4 | 80.0 | 60.1 |
| 3 months | 47.1 | 94.8 | 69.7 |

Strain UK355 (v3)

| | | | |
|---|---|---|---|
| Time zero | 2.7 | 4.0 | 5.0 |
| 2 months | 22.1 | 43.7 | 38/4 |
| 3 months | 21.3 | 55.0 | 41.7 |

Strain M1239 (v3)

| | | | |
|---|---|---|---|
| Time zero | 2.3 | 3.0 | 2.1 |
| 2 months | 5.0 | 15.7 | 5.9 |
| 3 months | 5.7 | 21.9 | 5.9 |

Strain M1239 (v3)

| | | | |
|---|---|---|---|
| Time zero | 1.2 | 1.6 | 1.1 |
| 2 months | 5.9 | 18.4 | 2.8 |
| 8 months | 1.9 | 4.1 | 1.6 |

Strain UK293 (v2)

| | | | |
|---|---|---|---|
| Time zero | 1.6 | 2.7 | 2.2 |
| 2 months | 9.2 | 52.0 | 7.0 |
| 8 months | 4.3 | 11.7 | 5.9 |

Strain UK414 (v2)

| | | | |
|---|---|---|---|
| Time zero | 1.4 | 2.1 | 1.6 |
| 2 months | 5.1 | 22.6 | 8.3 |
| 8 months | 3.1 | 10.9 | 6.3 |

Furthermore, the proportion of immunised subjects with a SBA titer above 1:8 was generally higher in group C compared to groups M and S e.g. 80% or more for strain M1239 after 3 doses compared to 50% or less in the other two groups.

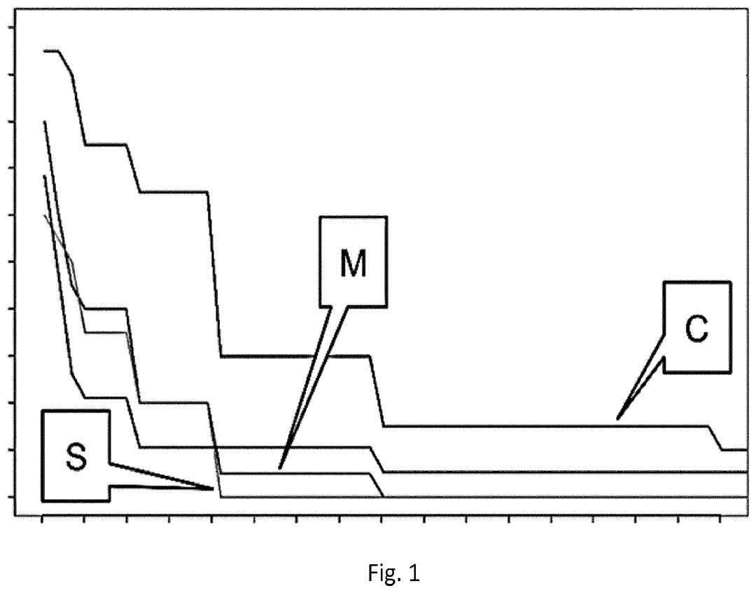

RCD curves (reverse cumulative distribution) of SBA titers also showed a better profile e.g. FIG. 1 shows a curve for 3 month sera against strain UK293, with group C being clearly above the others.

Pooled patient sera were used to assess coverage of a panel of 26 MenB strains which express a v2 or v3 fHbp. Again, titers (GMT) were highest in group C:

| | M | C | S |
|---|---|---|---|
| 3 months | 23 | 91 | 25 |
| 8 months | 7 | 43 | 9 |

These data thus show that vaccine ‘C’, in which the fHbp immunogen has been replaced and the OMV dosage was reduced 4-fold, is not inferior to the BEXSERO™ vaccine. Indeed, single-subject and pooled sera both show better seroresponse rates, higher GMTs, and increased strain coverage for vaccine ‘C’ when compared to the BEXSERO™ vaccine.

### Example 5: Antibody Avidity

Avidity of antibodies from patients in groups ‘C’ and ‘S’ was compared using a Gyrolab-based system which includes a wash step using a chaotropic agent to detach low affinity antibodies from antigen, giving in ‘Avidity Index’ as the percentage of high affinity anti-v1.fHbp antibodies out of total v1.fHbp-specific antibodies. Twenty separate sera were assessed 1 month after the first dose and 1 month after the third dose. In addition, SBA titres were assessed against strain H44/76, and correlations between avidity index and SBA titre (log 2) were determined.

Results (R and p by Pearson correlation) were as follows:

| | 1 month post-1 | | 1 month post-3 | |
|---|---|---|---|---|
| | R | p | R | p |
| C | 0.693 | 0.001 | 0.4667 | 0.0381 |
| S | 0.3565 | 0.1229 | 0.101 | 0.6718 |

Thus there was a significant correlation between SBA titre and avidity index in group ‘C’ at both time points, but not in group ‘S’. In subjects who received the vaccine with 12.5 μg/ml OMV the Avidity Index correlates with the SBA titres, which suggests that the presence of OMV has a positive impact on the quality of the induced antibodies. Overall, in subjects who received OMV the trend is that the bactericidal titers are higher and they correlate with the avidity of the antibodies induced by the vaccine formulation.

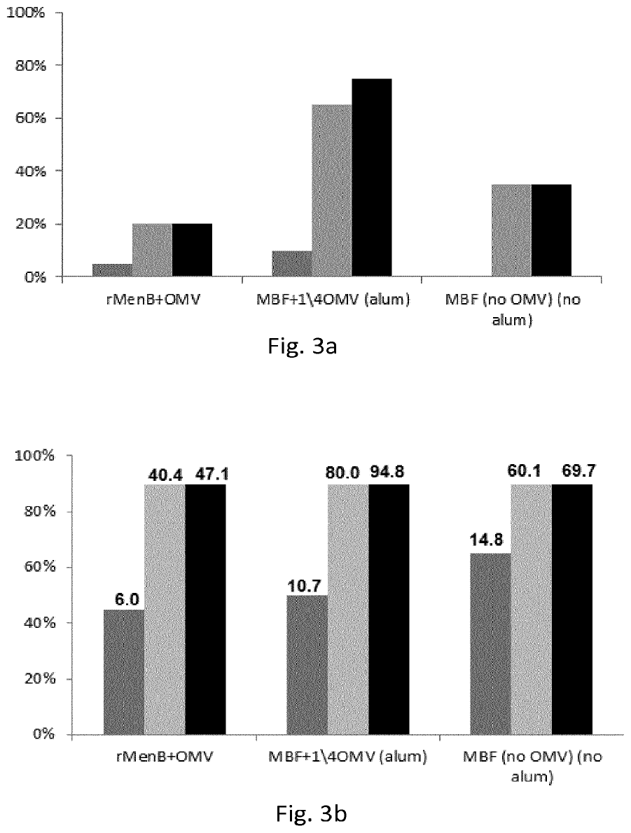

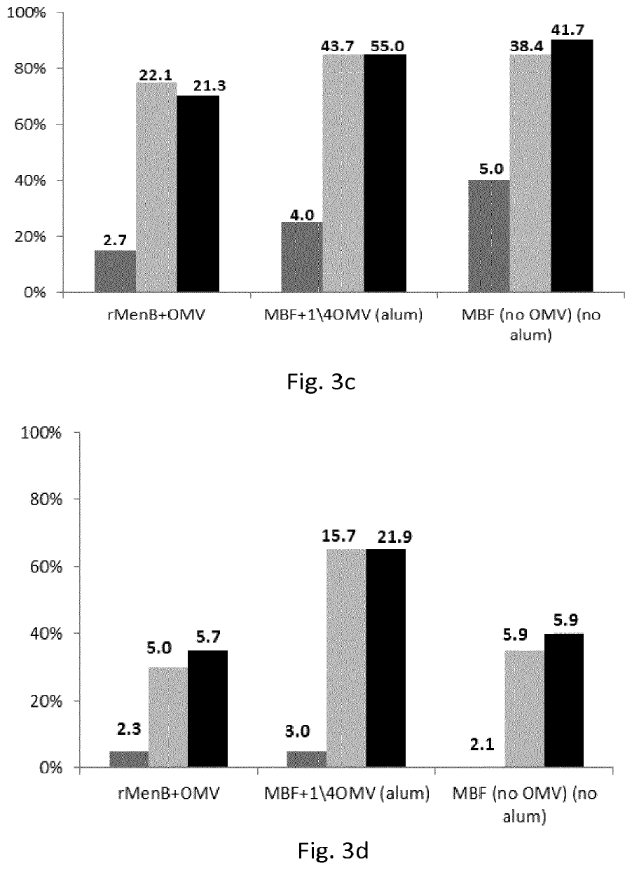

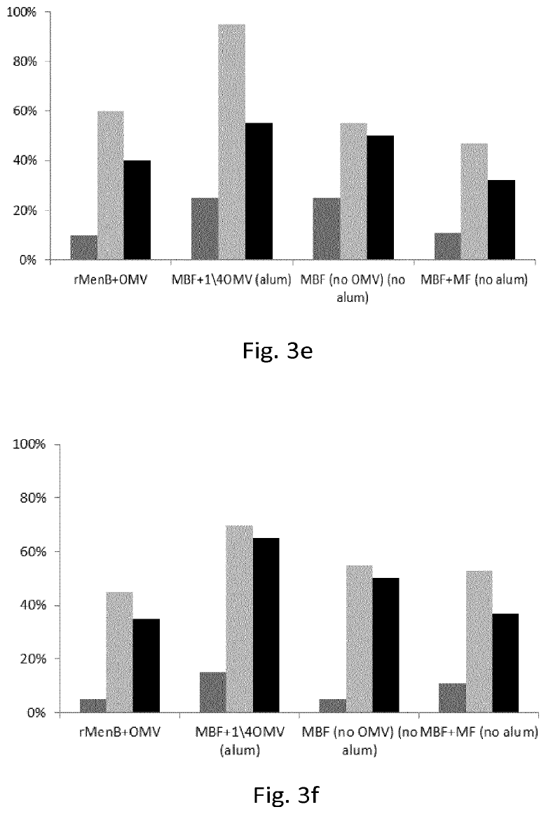

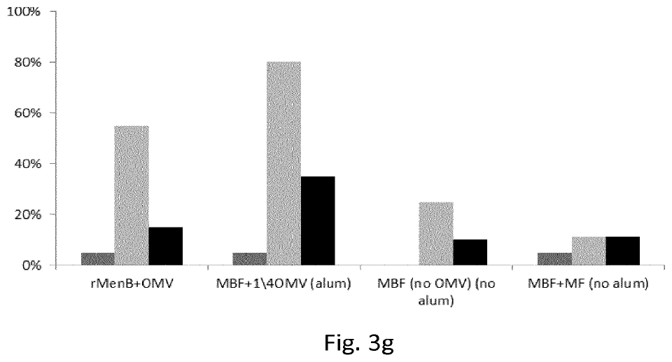

A subpanel of var2/3 strains was selected for single subject sera testing on the basis of following criteria: (i) Strains not covered by BEXSERO™ in previous clinical trials, (ii) Strains belong to relevant clonal complexes, (iii) Strains express epidemiologically relevant fHbp subvariants, (iv) Level of fHbp expression is medium, (v) Strains are specifically killed by 741-231 (competitive hSBA). Results are shown in FIGS. 3(a) to 3(g) DEMONSTRATING THAT 741-231+1/4OMV+ALUM ELICITS HIGHER GMT AGAINST THE 7 STRAINS tested. Thus, hSBA testing indicates that formulations including 741-231 fusion are not inferior to BEXSERO™. In fact, both single subject sera and pooled sera analysis on var2/3 strains show better seroresponse rates, higher GMT titers and increased strain coverage for formulation including 741-231+1/4OMV+alum.

### Example 6: Reduction of OMV Dosage and Use of 731 ‘S’ and 731 ‘SNB’

The BEXSERO™ product was modified by replacing the NMB2091-fHbp fusion polypeptide with the “triple fusion” stabilised or stabilised non-binding fHbp v2-v3-v1 polypeptides (SEQ ID NOs: 40 and 38 respectively) but also by reducing the OMV dosage to 10 μg/ml or 2.5 μg/ml:

| Group | Protein immunogens | | | OMVs |
|---|---|---|---|---|
| 1 | NMB2091-fHbp | NHBA-NMB1030 | NadA | 10 μg/ml |
| 2 | fHbp-v2-v3-v1 SNB | NHBA-NMB1030 | NadA | 2.5 μg/ml |
| 3 | fHbp-v2-v3-v1 S | NHBA-NMB1030 | NadA | 2.5 μg/ml |

To prepare mice antisera, 20 μg of NadA, NHBA-NMB1030 and either NMB2091-fHbp, fHbp 231S or fHbp 231SNB with 10 μg or 2.5 μg of OMV derived from strain NZ98/254 were used to immunize 6-week-old CDI female mice (Charles River). Eight mice per group were used. The antigens were administered intraperitoneally together with aluminium hydroxide (3 mg/ml) on days 0, 21 and 35.Sera were collected 2 weeks after the final bleed and heat-inactivated for 30 min at 56° C. before testing.

Serum Bactericidal Assay with Animal Sera and Human Complement

Serum bactericidal activity against Nm strains was evaluated as previously described. Human serum or plasma from a healthy adult (with no intrinsic bactericidal activity when tested at a final concentration of 25 or 50%) was used as a complement source. Serum bactericidal titers were defined as the serum dilution resulting in 50% decrease in colony forming units (CFU) per ml after 60 min incubation of bacteria with reaction mixture, compared to control CFU per ml at time 0.

The lowest dilution tested for each sera was 1:16 (limit of detection). Titers below the limit of detection were set to half that limit for the purposes of analysis and positive threshold was defined as a 4 fold rise compared to this value (i.e 32). Pooled serum derived from mice immunized with BEXSERO™ formulation were under the positive threshold for 14 strains among the 34 strain tested, while pooled sera derived from 2nd generation formulation were under the limit of detection for only 1 strain in case of vaccine formulation containing fHbp 231SNB and for 1 strains in case of formulation containin fHbp 231S.

hSBA data reported in the below table showed an increase of coverage elicited by the vaccine formulations containing fHbp 231S or fHbp 231SNB compare to BEXSERO™ in the panel of 34 strains tested:

| | | | hSBA results with different | | |
|---|---|---|---|---|---|
| | | | formulations | | |
| | | | | | 741-231 S + |
| MenB strains | | | | 741-231 SNB + | 961c + 287- |
| | | fHbp | | 961c + 287- | 953 + ¼ |
| | ID | subvariant | Bexsero | 953 + ¼ OMV | OMV |
| Bexsero | NVD000007 | 2.23 | >8192 | >8192 | >8192 |
| reference | NVD000005 | 2.16 | 2048 | 4096 | 2048 |
| strains | NVD000023 | 3.31 | 4096 | 4096 | 8192 |
| | NVD002240 | 2.553 | 32 | 512 | 128 |
| | NVD000025 | 1.1 | >8192 | >8192 | >8192 |
| | NVD001491 | 1.180 | 1024 | 1024 | 512 |
| | NVD000049 | 1.14 | 4096 | 4096 | 2048 |
| MenB strains | NVD001706 | 1.1 | 4096 | 4096 | 4096 |
| carrying var1 | NVD001889 | 1.4 | 1024 | 2048 | 2048 |
| fHbp | NVD001402 | 1.4 | 512 | 1024 | 1024 |
| | NVD001908 | 1.13 | 512 | 1024 | 1024 |
| | NVD001244 | 1.14 | 2048 | 2048 | 2048 |
| | NVD003213 | 1.15 | 2048 | 1024 | 2048 |
| | NVD001080 | 1.15 | 512 | 512 | 512 |
| | NVD000185 | 1.15 | 512 | 512 | 512 |
| | NVD000758 | 1.256 | <16 | 64 | <16 |
| MenB strains | NVD002368 | 2.16 | 64 | 1024 | 512 |
| carrying var2 | NVD002500 | 2.16 | <16 | 512 | 512 |
| fHbp | NVD000926 | 2.16 | 8192 | >8192 | 4096 |
| | NVD002552 | 2.19 | 16 | 512 | 1024 |
| | NVD001277 | 2.19 | <16 | 1024 | 2048 |
| | NVD001057 | 2.19 | 32 | 1024 | 512 |
| | NVD001342 | 2.19 | 64 | 2048 | 1024 |
| | NVD001391 | 2.19 | <16 | 512 | 512 |
| | NVD001288 | 2.21 | <16 | 512 | 512 |
| | NVD002690 | 2.24 | <16 | 256 | 256 |
| | NVD001287 | 2.24 | 16 | 128 | 256 |
| MenB strains | NVD000038 | 3.28 | <16 | 64 | 64 |
| carrying var3 | NVD000084 | 3.30 | <16 | 1024 | 2048 |
| fHbp | NVD003212 | 3.31 | <16 | 512 | 256 |
| | NVD003364 | 3.42 | <16 | 2048 | 2048 |
| | NVD002424 | 3.42 | <16 | 1024 | 1024 |
| | NVD003727 | 3.42 | <16 | <16 | <16 |

It will be understood that the invention is described above by way of example only and modifications may be made whilst remaining within the scope and spirit of the invention

### REFERENCES

[1] Carter (2013) BioDrugs 27:263-74.
[2] WO99/57280.
[3] Masignani et al. (2003) J Exp Med 197:789-799.
[4] Welsch et al. (2004) J Immunol 172:5605-15.
[5] Hou et al. (2005) J Infect Dis 192 (4): 580-90.
[6] WO03/063766.
[7] Fletcher et al. (2004) Infect Immun 72:2088-2100.
[8] Zhu et al. (2005) Infect Immun 73 (10): 6838-45.
[9] Cendron et al. (2011) Acta Crystallogr Sect F Struct Biol Cryst Commun. 67:531-5.
[10] Mascioni et al. (2009) J Biol Chem 284:8738-46.
[11] Pizza et al. (2008) Vaccine 26 Suppl 8:146-8.
[12] Malito et al. (2013) PNAS USA 110:3304-9.
[13] Marshall et al. (2012) Pediatr Infect Dis J 31:1061-8.
[14] McNeil et al. (2013) Microbiol Mol Biol Rev 77:234-52.
[15] Serruto et al. (2012) Vaccine 30 Suppl 2: B87-97.
[16] Scarselli et al. (2011) Sci Transl Med 3: 91ra62.
[17] Beernink et al. (2008) Infect Immun 76:4232-40.
[18] Scarselli et al. (2009) J Mol Biol 386:97-108.
[19] Giuntini et al. (2012) PLOS One 7: e34272.
[20] Vu et al. (2012) Sci Rep 2:341.
[21] Faleri et al. (2013) FASEB J fj. 13-239012.
[22] Beernink et al. (2011) J Immunol 186:3606-14.
[23] Tettelin et al. (2000) Science 287:1809-1815.
[24] WO00/66741.
[25] WO02/09643.
[26] Katial et al. (2002) Infect Immun 70:702-707.
[27] WO01/52885.
[28] European patent 0301992.
[29] Frasch et al. (2001) chapter 7 of Methods in Molecular Medicine, volume 66 (‘Meningococcal Vaccines: Methods and Protocols’, eds. Pollard & Maiden).
[30] Bjune et al. (1991) Lancet 338 (8775): 1093-1096.
[31] Fukasawa et al. (1999) Vaccine 17:2951-2958.
[32] WO02/09746.
[33] Rosenqvist et al. (1998) Dev. Biol. Stand. 92:323-333.
[34] European patent 0011243.
[35] Fredriksen et al. (1991) NIPH Ann. 14 (2): 67-80.
[36] WO01/91788.
[37] WO2004/019977.
[38] U.S. Pat. No. 6,558,677.
[39] WO01/09350.
[40] European patent 0449958.
[41] EP-A-0996712.
[42] EP-A-0680512.
[43] WO02/062378.
[44] WO99/59625.
[45] U.S. Pat. No. 6,180,111.
[46] WO01/34642.
[47] Peeters et al. (1996) Vaccine 14:1008-1015.
[48] Vermont et al. (2003) Infect Immun 71:1650-1655.
[49] WO2004/014417.
[50] WO2005/004908.
[51] WO2011/036562.
[52] Pizza et al. (2000) Science 287:1816-1820.
[53] WO2007/028408.
[54] (hypertext transfer protocol)://pubmlst.org/neisseria
[55] Budroni et al. (2011) PNAS USA 108:4494-99.
[56] Goldschneider et al. (1969) J. Exp. Med. 129:1307-26.
[57] Santos et al. (2001) Clinical and Diagnostic Laboratory Immunology 8:616-23.
[58] Frasch et al. (2009) Vaccine 27S: B112-6.
[59] Gennaro (2000) Remington: The Science and Practice of Pharmacy. 20th edition, ISBN: 0683306472.
[60] WO03/009869.
[61] Vaccine Design . . . (1995) eds. Powell & Newman. ISBN: 030644867X. Plenum.
[62] Giuliani et al. (2006) Proc Natl Acad Sci USA. 103: 10834-9.
[63] WO2004/032958.
[64] Costantino et al. (1992) Vaccine 10:691-698.
[65] Costantino et al. (1999) Vaccine 17:1251-1263.
[66] WO03/007985.
[67] Watson (2000) Pediatr Infect Dis J 19:331-332.
[68] Rubin (2000) Pediatr Clin North Am 47:269-285, v.
[69] Jedrzejas (2001) Microbiol Mol Biol Rev 65:187-207.
[710] Bell (2000) Pediatr Infect Dis J 19:1187-1188.
[71] Iwarson (1995) APMIS 103:321-326.
[72] Gerlich et al. (1990) Vaccine 8 Suppl: S63-68 & 79-80.
[73] Vaccines (1988) eds. Plotkin & Mortimer. ISBN 0-7216-1946-0.
[74] Del Guidice et al. (1998) Molecular Aspects of Medicine 19:1-70.
[75] Gustafsson et al. (1996) N. Engl. J. Med. 334:349-355.
[76] Rappuoli et al. (1991) TIBTECH 9:232-238.
[77] Sutter et al. (2000) Pediatr Clin North Am 47:287-308.
[78] Zimmerman & Spann (1999) Am Fam Physician 59:113-118, 125-126.
[79] McMichael (2000) Vaccine 19 Suppl 1: S101-107.
[80] Schuchat (1999) Lancet 353 (9146): 51-6.
[81] WO02/34771.
[82] Dale (1999) Infect Dis Clin North Am 13:227-43, viii.
[83] Ferretti et al. (2001) PNAS USA 98:4658-4663.
[84] Kuroda et al. (2001) Lancet 357 (9264): 1225-1240; see also pages 1218-1219.
[85] Jones (2001) Curr Opin Investig Drugs 2:47-49.
[86] Ravenscroft et al. (1999) Vaccine 17:2802-2816.
[87] Research Disclosure, 453077 (January 2002).
[88] EP-A-0372501.
[89] EP-A-0378881.
[90] EP-A-0427347.
[91] WO93/17712.
[92] WO94/03208.
[93] WO98/58668.
[94] EP-A-0471177.
[95] WO91/01146.
[96] Falugi et al. (2001) Eur J Immunol 31:3816-3824.
[97] Baraldo et al. (2004) Infect Immun 72 (8): 4884-7.
[98] EP-A-0594610.
[99] Ruan et al. (1990) J Immunol 145:3379-3384.
[100] WO00/56360.
[101] Kuo et al. (1995) Infect Immun 63:2706-13.
[102] Michon et al. (1998) Vaccine. 16:1732-41.
[103] WO02/091998.
[104] WO01/72337.
[105] WO00/61761.
[106] WO00/33882
[107] Lees et al. (1996) Vaccine 14:190-198.
[108] WO95/08348.
[109] U.S. Pat. No. 4,882,317
[110] U.S. Pat. No. 4,695,624
[111] Porro et al. (1985) Mol Immunol 22:907-919.s
[112] EP-A-0208375
[113] WO00/10599
[114] Gever et al. Med. Microbiol. Immunol, 165:171-288 (1979).
[115] U.S. Pat. No. 4,057,685.
[116] U.S. Pat. No. 4,673,574; 4,761,283; 4,808,700.
[117] U.S. Pat. No. 4,459,286.
[118] U.S. Pat. No. 4,965,338
[119] U.S. Pat. No. 4,663,160.
[120] U.S. Pat. No. 4,761,283
[121] U.S. Pat. No. 4,356,170
[122] Needleman & Wunsch (1970) J. Mol. Biol. 48, 443-453.
[123] Rice et al. (2000) Trends Genet 16:276-277.

SEQUENCE LISTING

>SEQ ID NO: 1 [MC58, v1]
MNRTAFCCLSLTTALILTACSSGGGGVAADIGAGLADALTAPLDHKDKGLQSLTLDQSVRKNEKL
KLAAQGAEKTYGNGDSLNTGKLKNDKVSRFDFIRQIEVDGQLITLESGEFQVYKQSHSALTAFQT
EQIQDSEHSGKMVAKRQFRIGDIAGEHTSFDKLPEGGRATYRGTAFGSDDAGGKLTYTIDFAAKQ
GNGKIEHLKSPELNVDLAAADIKPDGKRHAVISGSVLYNQAEKGSYSLGIFGGKAQEVAGSAEVK
TVNGIRHIGLAAKQ
>SEQ ID NO: 2 [2996, v2]
MNRTAFCCLSLTAALILTACSSGGGGVAADIGAGLADALTAPLDHKDKSLQSLTLDQSVRKNEKL
KLAAQGAEKTYGNGDSLNTGKLKNDKVSRFDFIRQIEVDGQLITLESGEFQIYKQDHSAVVALQI
EKINNPDKIDSLINQRSFLVSGLGGEHTAFNQLPDGKAEYHGKAFSSDDAGGKLTYTIDFAAKQG
HGKIEHLKTPEQNVELAAAELKADEKSHAVILGDTRYGSEEKGTYHLALFGDRAQEIAGSATVKI
GEKVHEIGIAGKQ
>SEQ ID NO: 3 [M1239, v3]
MNRTAFCCLSLTTALILTACSSGGGGSGGGGVAADIGTGLADALTAPLDHKDKGLKSLTLEDSIP
QNGTLTLSAQGAEKTFKAGDKDNSLNTGKLKNDKISRFDFVQKIEVDGQTITLASGEFQIYKQNH
SAVVALQIEKINNPDKTDSLINQRSFLVSGLGGEHTAFNQLPGGKAEYHGKAFSSDDPNGRLHYS
IDFTKKQGYGRIEHLKTLEQNVELAAAELKADEKSHAVILGDTRYGSEEKGTYHLALFGDRAQEI
AGSATVKIGEKVHEIGIAGKQ
>SEQ ID NO: 4 [MC58, v1, mature]
CSSGGGGVAADIGAGLADALTAPLDHKDKGLQSLTLDQSVRKNEKLKLAAQGAEKTYGNGDSLNT
GKLKNDKVSRFDFIRQIEVDGQLITLESGEFQVYKQSHSALTAFQTEQIQDSEHSGKMVAKRQER
IGDIAGEHTSFDKLPEGGRATYRGTAFGSDDAGGKLTYTIDFAAKQGNGKIEHLKSPELNVDLAA
ADIKPDGKRHAVISGSVLYNQAEKGSYSLGIFGGKAQEVAGSAEVKTVNGIRHIGLAAKQ
>SEQ ID NO: 5 [2996 mature]
CSSGGGGVAADIGAGLADALTAPLDHKDKSLQSLTLDQSVRKNEKLKLAAQGAEKTYGNGDSLNT
GKLKNDKVSRFDFIRQIEVDGQLITLESGEFQIYKQDHSAVVALQIEKINNPDKIDSLINQRSFL
VSGLGGEHTAFNQLPDGKAEYHGKAFSSDDAGGKLTYTIDFAAKQGHGKIEHLKTPEQNVELAAA
ELKADEKSHAVILGDTRYGSEEKGTYHLALFGDRAQEIAGSATVKIGEKVHEIGIAGKQ
>SEQ ID NO: 6 [M1239, mature]
CSSGGGGSGGGGVAADIGTGLADALTAPLDHKDKGLKSLTLEDSIPQNGTLTLSAQGAEKTEKAG
DKDNSLNTGKLKNDKISRFDFVQKIEVDGQTITLASGEFQIYKQNHSAVVALQIEKINNPDKTDS
LINQRSFLVSGLGGEHTAFNQLPGGKAEYHGKAFSSDDPNGRLHYSIDFTKKQGYGRIEHLKTLE
QNVELAAAELKADEKSHAVILGDTRYGSEEKGTYHLALFGDRAQEIAGSATVKIGEKVHEIGIAG
KQ
>SEQ ID NO: 7 [MC58, ΔG]
VAADIGAGLADALTAPLDHKDKGLQSLTLDQSVRKNEKLKLAAQGAEKTYGNGDSLNTGKLKNDK
VSRFDFIRQIEVDGQLITLESGEFQVYKQSHSALTAFQTEQIQDSEHSGKMVAKRQFRIGDIAGE
HTSFDKLPEGGRATYRGTAFGSDDAGGKLTYTIDFAAKQGNGKIEHLKSPELNVDLAAADIKPDG
KRHAVISGSVLYNQAEKGSYSLGIFGGKAQEVAGSAEVKTVNGIRHIGLAAKQ
>SEQ ID NO: 8 [2996 ΔG]
VAADIGAGLADALTAPLDHKDKSLQSLTLDQSVRKNEKLKLAAQGAEKTYGNGDSLNTGKLKNDK
VSRFDFIRQIEVDGQLITLESGEFQIYKQDHSAVVALQIEKINNPDKIDSLINQRSFLVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHEIGIAGKQ
>SEQ ID NO: 9 [M1239, ΔG]
VAADIGTGLADALTAPLDHKDKGLKSLTLEDSIPQNGTLTLSAQGAEKTFKAGDKDNSLNTGKLK
NDKISRFDFVQKIEVDGQTITLASGEFQIYKQNHSAVVALQIEKINNPDKTDSLINQRSFLVSGL
GGEHTAFNQLPGGKAEYHGKAFSSDDPNGRLHYSIDFTKKQGYGRIEHLKTLEQNVELAAAELKA
DEKSHAVILGDTRYGSEEKGTYHLALFGDRAQEIAGSATVKIGEKVHEIGIAGKQ
>SEQ ID NO: 10 [fHbp fusion polypeptide]
MGPDSDRLQQRRVAADIGAGLADALTAPLDHKDKSLQSLTLDQSVRKNEKLKLAAQGAEKTYGNG
DSLNTGKLKNDKVSRFDFIRQIEVDGQLITLESGEFQIYKQDHSAVVALQIEKINNPDKIDSLIN
QRSFLVSGLGGEHTAFNQLPDGKAEYHGKAFSSDDAGGKLTYTIDFAAKQGHGKIEHLKTPEQNV
ELAAAELKADEKSHAVILGDTRYGSEEKGTYHLALFGDRAQEIAGSATVKIGEKVHEIGIAGKQG
SGPDSDRLQQRRVAADIGTGLADALTAPLDHKDKGLKSLTLEDSIPQNGTLTLSAQGAEKTFKAG
DKDNSLNTGKLKNDKISRFDFVQKIEVDGQTITLASGEFQIYKQNHSAVVALQIEKINNPDKTDS
LINQRSFLVSGLGGEHTAFNQLPGGKAEYHGKAFSSDDPNGRLHYSIDFTKKQGYGRIEHLKTLE
QNVELAAAELKADEKSHAVILGDTRYGSEEKGTYHLALFGDRAQEIAGSATVKIGEKVHEIGIAG
KQGSGGGGVAADIGAGLADALTAPLDHKDKGLQSLTLDQSVRKNEKLKLAAQGAEKTYGNGDSLN
TGKLKNDKVSRFDFIRQIEVDGQLITLESGEFQVYKQSHSALTAFQTEQIQDSEHSGKMVAKRQF
RIGDIAGEHTSFDKLPEGGRATYRGTAFGSDDAGGKLTYTIDFAAKQGNGKIEHLKSPELNVDLA
AADIKPDGKRHAVISGSVLYNQAEKGSYSLGIFGGKAQEVAGSAEVKTVNGIRHIGLAAKQ
>SEQ ID NO: 11 [NHBA, MC58]
MFKRSVIAMACIFALSACGGGGGGSPDVKSADTLSKPAAPVVSEKETEAKEDAPQAGSQGQGAPS
AQGSQDMAAVSEENTGNGGAVTADNPKNEDEVAQNDMPQNAAGTDSSTPNHTPDPNMLAGNMENQ
ATDAGESSQPANQPDMANAADGMQGDDPSAGGQNAGNTAAQGANQAGNNQAAGSSDPIPASNPAP
ANGGSNFGRVDLANGVLIDGPSQNITLTHCKGDSCSGNNFLDEEVQLKSEFEKLSDADKISNYKK
DGKNDKFVGLVADSVQMKGINQYIIFYKPKPTSFARFRRSARSRRSLPAEMPLIPVNQADTLIVD
GEAVSLTGHSGNIFAPEGNYRYLTYGAEKLPGGSYALRVQGEPAKGEMLAGAAVYNGEVLHFHTE
NGRPYPTRGRFAAKVDFGSKSVDGIIDSGDDLHMGTQKFKAAIDGNGFKGTWTENGSGDVSGKFY
GPAGEEVAGKYSYRPTDAEKGGFGVFAGKKEQD
>SEQ ID NO: 12 [NHBA fragment]
SPDVKSADTLSKPAAPVVSEKETEAKEDAPQAGSQGQGAPSAQGSQDMAAVSEENTGNGGAVTAD
NPKNEDEVAQNDMPQNAAGTDSSTPNHTPDPNMLAGNMENQATDAGESSQPANQPDMANAADGMQ
GDDPSAGGQNAGNTAAQGANQAGNNQAAGSSDPIPASNPAPANGGSNFGRVDLANGVLIDGPSQN
ITLTHCKGDSCSGNNFLDEEVQLKSEFEKLSDADKISNYKKDGKNDKFVGLVADSVQMKGINQYI
IFYKPKPTSFARFRRSARSRRSLPAEMPLIPVNQADTLIVDGEAVSLTGHSGNIFAPEGNYRYLT
YGAEKLPGGSYALRVQGEPAKGEMLAGAAVYNGEVLHFHTENGRPYPTRGRFAAKVDFGSKSVDG
IIDSGDDLHMGTQKFKAAIDGNGFKGTWTENGSGDVSGKFYGPAGEEVAGKYSYRPTDAEKGGFG
VFAGKKEQD
>SEQ ID NO: 13 [NHBA mature]
CGGGGGGSPDVKSADTLSKPAAPVVSEKETEAKEDAPQAGSQGQGAPSAQGSQDMAAVSEENTGN
GGAVTADNPKNEDEVAQNDMPQNAAGTDSSTPNHTPDPNMLAGNMENQATDAGESSQPANQPDMA
NAADGMQGDDPSAGGQNAGNTAAQGANQAGNNQAAGSSDPIPASNPAPANGGSNFGRVDLANGVL
IDGPSQNITLTHCKGDSCSGNNFLDEEVQLKSEFEKLSDADKISNYKKDGKNDKFVGLVADSVQM
KGINQYIIFYKPKPTSFARFRRSARSRRSLPAEMPLIPVNQADTLIVDGEAVSLTGHSGNIFAPE
GNYRYLTYGAEKLPGGSYALRVQGEPAKGEMLAGAAVYNGEVLHFHTENGRPYPTRGRFAAKVDE
GSKSVDGIIDSGDDLHMGTQKFKAAIDGNGFKGTWTENGSGDVSGKFYGPAGEEVAGKYSYRPTD
AEKGGFGVFAGKKEQD
>SEQ ID NO: 14 [NMB1030, MC58]
MKKIIFAALAAAAISTASAATYKVDEYHANARFAIDHENTSTNVGGFYGLTGSVEFDQAKRDGKI
DITIPIANLQSGSQHFTDHLKSADIFDAAQYPDIRFVSTKFNFNGKKLVSVDGNLTMHGKTAPVK
LKAEKFNCYQSPMEKTEVCGGDFSTTIDRTKWGMDYLVNVGMTKSVRIDIQIEAAKQ
>SEQ ID NO: 15 [NMB1030 fragment]
ATYKVDEYHANARFAIDHFNTSTNVGGFYGLTGSVEFDQAKRDGKIDITIPIANLQSGSQHFTDH
LKSADIFDAAQYPDIRFVSTKFNFNGKKLVSVDGNLTMHGKTAPVKLKAEKENCYQSPMEKTEVC
GGDFSTTIDRTKWGMDYLVNVGMTKSVRIDIQIEAAKQ
>SEQ ID NO: 16 [NHBA fusion]
MASPDVKSADTLSKPAAPVVSEKETEAKEDAPQAGSQGQGAPSAQGGQDMAAVSEENTGNGGAAA
TDKPKNEDEGAQNDMPQNAADTDSLTPNHTPASNMPAGNMENQAPDAGESEQPANQPDMANTADG
MQGDDPSAGGENAGNTAAQGTNQAENNQTAGSQNPASSTNPSATNSGGDFGRTNVGNSVVIDGPS
QNITLTHCKGDSCSGNNFLDEEVQLKSEFEKLSDADKISNYKKDGKNDGKNDKFVGLVADSVQMK
GINQYIIFYKPKPTSFARFRRSARSRRSLPAEMPLIPVNQADTLIVDGEAVSLTGHSGNIFAPEG
NYRYLTYGAEKLPGGSYALRVQGEPSKGEMLAGTAVYNGEVLHFHTENGRPSPSRGRFAAKVDFG
SKSVDGIIDSGDGLHMGTQKFKAAIDGNGFKGTWTENGGGDVSGKFYGPAGEEVAGKYSYRPTDA
EKGGFGVFAGKKEQDGSGGGGATYKVDEYHANARFAIDHFNTSTNVGGFYGLTGSVEFDQAKRDG
KIDITIPVANLQSGSQHFTDHLKSADIFDAAQYPDIRFVSTKFNFNGKKLVSVDGNLTMHGKTAP
VKLKAEKFNCYQSPMAKTEVCGGDFSTTIDRTKWGVDYLVNVGMTKSVRIDIQIEAAKQ
>SEQ ID NO: 17 [NadA, MC58]
MSMKHFPSKVLTTAILATFCSGALAATSDDDVKKAATVAIVAAYNNGQEINGFKAGETIYDIGED
GTITQKDATAADVEADDFKGLGLKKVVTNLTKTVNENKQNVDAKVKAAESEIEKLTTKLADTDAA
LADTDAALDETTNALNKLGENITTFAEETKTNIVKIDEKLEAVADTVDKHAEAFNDIADSLDETN
TKADEAVKTANEAKQTAEETKQNVDAKVKAAETAAGKAEAAAGTANTAADKAEAVAAKVTDIKAD
IATNKADIAKNSARIDSLDKNVANLRKETRQGLAEQAALSGLFQPYNVGRFNVTAAVGGYKSESA
VAIGTGFRFTENFAAKAGVAVGTSSGSSAAYHVGVNYEW
>SEQ ID NO: 18 [NadA]
LAATSDDDVKKAATVAIVAAYNNGQEINGFKAGETIYDIGEDGTITQKDATAADVEADDEKGLGL
KKVVTNLTKTVNENKQNVDAKVKAAESEIEKLTTKLADTDAALADTDAALDETTNALNKLGENIT
TFAEETKTNIVKIDEKLEAVADTVDKHAEAFNDIADSLDETNTKADEAVKTANEAKQTAEETKQN
VDAKVKAAETAAGKAEAAAGTANTAADKAEAVAAKVTDIKADIATNKADIAKNSARIDSLDKNVA
NLRKETRQGLAEQAALSGLFQPYNVGRENVTAAVGGYKSESAVAIGTGFRFTENFAAKAGVAVGT
SSGSSAAYHVGVNYEW
>SEQ ID NO: 19 [NadA fragment]
ATNDDDVKKAATVAIAAAYNNGQEINGFKAGETIYDIDEDGTITKKDATAADVEADDFKGLGLKK
VVTNLTKTVNENKQNVDAKVKAAESEIEKLTTKLADTDAALADTDAALDATTNALNKLGENITTF
AEETKTNIVKIDEKLEAVADTVDKHAEAFNDIADSLDETNTKADEAVKTANEAKQTAEETKQNVD
AKVKAAETAAGKAEAAAGTANTAADKAEAVAAKVTDIKADIATNKDNIAKKANSADVYTREESDS
KFVRIDGLNATTEKLDTRLASAEKSIADHDTRLNGLDKTVSDLRKETRQGLAEQAALSGLFQPYN
VG
>SEQ ID NO: 20 [NMB2091, MC58]
MKPKPHTVRTLIAAIFSLALSGCVSAVIGSAAVGAKSAVDRRTTGAQTDDNVMALRIETTARSYL
RQNNQTKGYTPQISVVGYNRHLLLLGQVATEGEKQFVGQIARSEQAAEGVYNYITVASLPRTAGD
IAGDTWNTSKVRATLLGISPATQARVKIVTYGNVTYVMGILTPEEQAQITQKVSTTVGVQKVITL
YQNYVQR
>SEQ ID NO: 21 [NMB2091]
SAVIGSAAVGAKSAVDRRTTGAQTDDNVMALRIETTARSYLRQNNQTKGYTPQISVVGYDRHLLL
LGQVATEGEKQFVGQIARSEQAAEGVYNYITVASLPRTAGDIAGDTWNTSKVRATLLGISPATRA
RVKIVTYGNVTYVMGILTPEEQAQITQKVSTTVGVQKVITLYQNYVQR
>SEQ ID NO: 22 [linker]
GSGGGG
>SEQ ID NO: 23 [linker]
GPDSDRLQQRR
>SEQ ID NO: 24 [linker]
GSGPDSDRLQQRR
>SEQ ID NO: 25 [linker]
GKGPDSDRLQQRR
>SEQ ID NO: 26 [N-terminal sequence]
MGPDSDRLQQRR
>SEQ ID NO: 27 [N-terminal sequence]
MAS
>SEQ ID NO: 28 [linker]
LEHHHHHH
>SEQ ID NO: 29 [fHbp fusion polypeptide]
MGPDSDRLQQRRVAADIGAGLADALTAPLDHKDKSLQSLTLDQSVRKNEKLKLAAQGAEKTYGNG
DSLNTGKLKNDKVSRFDFIRQIEVDGQLITLESGEFQIYKQDHSAVVALQIEKINNPDKIDSLIN
QRSFLVSGLGGEHTAFNQLPDGKAEYHGKAFSSDDAGGKLTYTIDFAAKQGHGKIEHLKTPEQNV
ELAAAELKADEKSHAVILGDTRYGSEEKGTYHLALFGDRAQEIAGSATVKIGEKVHEIGIAGKQG
SGGGGVAADIGTGLADALTAPLDHKDKGLKSLTLEDSIPQNGTLTLSAQGAEKTFKAGDKDNSLN
TGKLKNDKISRFDFVQKIEVDGQTITLASGEFQIYKQNHSAVVALQIEKINNPDKTDSLINQRSF
LVSGLGGEHTAFNQLPGGKAEYHGKAFSSDDPNGRLHYSIDFTKKQGYGRIEHLKTLEQNVELAA
AELKADEKSHAVILGDTRYGSEEKGTYHLALFGDRAQEIAGSATVKIGEKVHEIGIAGKQGSGGG
GVAADIGAGLADALTAPLDHKDKGLQSLTLDQSVRKNEKLKLAAQGAEKTYGNGDSLNTGKLKND
KVSRFDFIRQIEVDGQLITLESGEFQVYKQSHSALTAFQTEQIQDSEHSGKMVAKRQFRIGDIAG
EHTSFDKLPEGGRATYRGTAFGSDDAGGKLTYTIDFAAKQGNGKIEHLKSPELNVDLAAADIKPD
GKRHAVISGSVLYNQAEKGSYSLGIFGGKAQEVAGSAEVKTVNGIRHIGLAAKQ
>SEQ ID NO: 30 [fHbp fusion polypeptide]
VAADIGAGLADALTAPLDHKDKSLQSLTLDQSVRKNEKLKLAAQGAEKTYGNGDSLNTGKLKNDK
VSRFDFIRQIEVDGQLITLESGEFQIYKQDHSAVVALQIEKINNPDKIDSLINQRSFLVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHEIGIAGKQGSGGGGVAADIGT
GLADALTAPLDHKDKGLKSLTLEDSIPQNGTLTLSAQGAEKTFKAGDKDNSLNTGKLKNDKISRF
DFVQKIEVDGQTITLASGEFQIYKQNHSAVVALQIEKINNPDKTDSLINQRSFLVSGLGGEHTAF
NQLPGGKAEYHGKAFSSDDPNGRLHYSIDFTKKQGYGRIEHLKTLEQNVELAAAELKADEKSHAV
ILGDTRYGSEEKGTYHLALFGDRAQEIAGSATVKIGEKVHEIGIAGKQGSGGGGVAADIGAGLAD
ALTAPLDHKDKGLQSLTLDQSVRKNEKLKLAAQGAEKTYGNGDSLNTGKLKNDKVSREDFIRQIE
VDGQLITLESGEFQVYKQSHSALTAFQTEQIQDSEHSGKMVAKRQFRIGDIAGEHTSFDKLPEGG
RATYRGTAFGSDDAGGKLTYTIDFAAKQGNGKIEHLKSPELNVDLAAADIKPDGKRHAVISGSVL
YNQAEKGSYSLGIFGGKAQEVAGSAEVKTVNGIRHIGLAAKQ
>SEQ ID NO: 31 [fHbp fusion polypeptide, fH binding disrupted]
Where X at residue 240 is any amino acid other than E, X at residue 496
is any amino acid other than E, and X at residue 543 is any amino acid
other than R.
VAADIGAGLADALTAPLDHKDKSLQSLTLDQSVRKNEKLKLAAQGAEKTYGNGDSLNTGKLKNDK
VSRFDFIRQIEVDGQLITLESGEFQIYKQDHSAVVALQIEKINNPDKIDSLINQRSFLVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHXIGIAGKQGSGGGGVAADIGT
GLADALTAPLDHKDKGLKSLTLEDSIPQNGTLTLSAQGAEKTFKAGDKDNSLNTGKLKNDKISRF
DFVQKIEVDGQTITLASGEFQIYKQNHSAVVALQIEKINNPDKTDSLINQRSFLVSGLGGEHTAF
NQLPGGKAEYHGKAFSSDDPNGRLHYSIDFTKKQGYGRIEHLKTLEQNVELAAAELKADEKSHAV
ILGDTRYGSEEKGTYHLALFGDRAQEIAGSATVKIGEKVHXIGIAGKQGSGGGGVAADIGAGLAD
ALTAPLDHKDKGLQSLTLDQSVXKNEKLKLAAQGAEKTYGNGDSLNTGKLKNDKVSRFDFIRQIE
VDGQLITLESGEFQVYKQSHSALTAFQTEQIQDSEHSGKMVAKRQFRIGDIAGEHTSEDKLPEGG
RATYRGTAFGSDDAGGKLTYTIDFAAKQGNGKIEHLKSPELNVDLAAADIKPDGKRHAVISGSVL
YNQAEKGSYSLGIFGGKAQEVAGSAEVKTVNGIRHIGLAAKQ
>SEQ ID NO: 32 [fHbp fusion polypeptide, fH binding disrupted]
VAADIGAGLADALTAPLDHKDKSLQSLTLDQSVRKNEKLKLAAQGAEKTYGNGDSLNTGKLKNDK
VSRFDFIRQIEVDGQLITLESGEFQIYKQDHSAVVALQIEKINNPDKIDSLINQRSFLVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHAIGIAGKQGSGGGGVAADIGT
GLADALTAPLDHKDKGLKSLTLEDSIPQNGTLTLSAQGAEKTFKAGDKDNSLNTGKLKNDKISRF
DFVQKIEVDGQTITLASGEFQIYKQNHSAVVALQIEKINNPDKTDSLINQRSFLVSGLGGEHTAF
NQLPGGKAEYHGKAFSSDDPNGRLHYSIDFTKKQGYGRIEHLKTLEQNVELAAAELKADEKSHAV
ILGDTRYGSEEKGTYHLALFGDRAQEIAGSATVKIGEKVHAIGIAGKQGSGGGGVAADIGAGLAD
ALTAPLDHKDKGLQSLTLDQSVSKNEKLKLAAQGAEKTYGNGDSLNTGKLKNDKVSREDFIRQIE
VDGQLITLESGEFQVYKQSHSALTAFQTEQIQDSEHSGKMVAKRQFRIGDIAGEHTSFDKLPEGG
RATYRGTAFGSDDAGGKLTYTIDFAAKQGNGKIEHLKSPELNVDLAAADIKPDGKRHAVISGSVL
YNQAEKGSYSLGIFGGKAQEVAGSAEVKTVNGIRHIGLAAKQ
>SEQ ID NO: 33 [fHbp fusion polypeptide, stabilised]
Where X at residue 32 is any amino acid other than S, X at residue 123 is
any amino acid other than L, X at residue 285 is any amino acid other than
S, and X at residue 379 is any amino acid other than L.
VAADIGAGLADALTAPLDHKDKSLQSLTLDQXVRKNEKLKLAAQGAEKTYGNGDSLNTGKLKNDK
VSRFDFIRQIEVDGQLITLESGEFQIYKQDHSAVVALQIEKINNPDKIDSLINQRSFXVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHEIGIAGKQGSGGGGVAADIGT
GLADALTAPLDHKDKGLKSLTLEDXIPQNGTLTLSAQGAEKTFKAGDKDNSLNTGKLKNDKISRF
DFVQKIEVDGQTITLASGEFQIYKQNHSAVVALQIEKINNPDKTDSLINQRSFXVSGLGGEHTAF
NQLPGGKAEYHGKAFSSDDPNGRLHYSIDFTKKQGYGRIEHLKTLEQNVELAAAELKADEKSHAV
ILGDTRYGSEEKGTYHLALFGDRAQEIAGSATVKIGEKVHEIGIAGKQGSGGGGVAADIGAGLAD
ALTAPLDHKDKGLQSLTLDQSVRKNEKLKLAAQGAEKTYGNGDSLNTGKLKNDKVSREDFIRQIE
VDGQLITLESGEFQVYKQSHSALTAFQTEQIQDSEHSGKMVAKRQFRIGDIAGEHTSFDKLPEGG
RATYRGTAFGSDDAGGKLTYTIDFAAKQGNGKIEHLKSPELNVDLAAADIKPDGKRHAVISGSVL
YNQAEKGSYSLGIFGGKAQEVAGSAEVKTVNGIRHIGLAAKQ
>SEQ ID NO: 34 [fHbp fusion polypeptide, stabilised]
VAADIGAGLADALTAPLDHKDKSLQSLTLDQVVRKNEKLKLAAQGAEKTYGNGDSLNTGKLKNDK
VSRFDFIRQIEVDGQLITLESGEFQIYKQDHSAVVALQIEKINNPDKIDSLINQRSFRVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHEIGIAGKQGSGGGGVAADIGT
GLADALTAPLDHKDKGLKSLTLEDVIPQNGTLTLSAQGAEKTFKAGDKDNSLNTGKLKNDKISRF
DFVQKIEVDGQTITLASGEFQIYKQNHSAVVALQIEKINNPDKTDSLINQRSFRVSGLGGEHTAF
NQLPGGKAEYHGKAFSSDDPNGRLHYSIDFTKKQGYGRIEHLKTLEQNVELAAAELKADEKSHAV
ILGDTRYGSEEKGTYHLALFGDRAQEIAGSATVKIGEKVHEIGIAGKQGSGGGGVAADIGAGLAD
ALTAPLDHKDKGLQSLTLDQSVRKNEKLKLAAQGAEKTYGNGDSLNTGKLKNDKVSREDFIRQIE
VDGQLITLESGEFQVYKQSHSALTAFQTEQIQDSEHSGKMVAKRQFRIGDIAGEHTSFDKLPEGG
RATYRGTAFGSDDAGGKLTYTIDFAAKQGNGKIEHLKSPELNVDLAAADIKPDGKRHAVISGSVL
YNQAEKGSYSLGIFGGKAQEVAGSAEVKTVNGIRHIGLAAKQ
>SEQ ID NO: 35 [fHbp fusion polypeptide, stabilised & R41]
Where X at residue 32 is any amino acid other thanS , X at residue 123 is
any amino acid other than L, X at residue 285 is any amino acid other than
S, X at residue 379 is any amino acid other than L, and X at residue 543 is
any amino acid other than R.
VAADIGAGLADALTAPLDHKDKSLQSLTLDQXVRKNEKLKLAAQGAEKTYGNGDSLNTGKLKNDK
VSRFDFIRQIEVDGQLITLESGEFQIYKQDHSAVVALQIEKINNPDKIDSLINQRSFXVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHEIGIAGKQGSGGGGVAADIGT
GLADALTAPLDHKDKGLKSLTLEDXIPQNGTLTLSAQGAEKTFKAGDKDNSLNTGKLKNDKISRF
DFVQKIEVDGQTITLASGEFQIYKQNHSAVVALQIEKINNPDKTDSLINQRSFXVSGLGGEHTAF
NQLPGGKAEYHGKAFSSDDPNGRLHYSIDFTKKQGYGRIEHLKTLEQNVELAAAELKADEKSHAV
ILGDTRYGSEEKGTYHLALFGDRAQEIAGSATVKIGEKVHEIGIAGKQGSGGGGVAADIGAGLAD
ALTAPLDHKDKGLQSLTLDQSVXKNEKLKLAAQGAEKTYGNGDSLNTGKLKNDKVSREDFIRQIE
VDGQLITLESGEFQVYKQSHSALTAFQTEQIQDSEHSGKMVAKRQFRIGDIAGEHTSFDKLPEGG
RATYRGTAFGSDDAGGKLTYTIDFAAKQGNGKIEHLKSPELNVDLAAADIKPDGKRHAVISGSVL
YNQAEKGSYSLGIFGGKAQEVAGSAEVKTVNGIRHIGLAAKQ
>SEQ ID NO: 36 [fHbp fusion polypeptide, stabilised & R41S]
VAADIGAGLADALTAPLDHKDKSLQSLTLDQVVRKNEKLKLAAQGAEKTYGNGDSLNTGKLKNDK
VSRFDFIRQIEVDGQLITLESGEFQIYKQDHSAVVALQIEKINNPDKIDSLINQRSFRVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHEIGIAGKQGSGGGGVAADIGT
GLADALTAPLDHKDKGLKSLTLEDVIPQNGTLTLSAQGAEKTFKAGDKDNSLNTGKLKNDKISRF
DFVQKIEVDGQTITLASGEFQIYKQNHSAVVALQIEKINNPDKTDSLINQRSFRVSGLGGEHTAF
NQLPGGKAEYHGKAFSSDDPNGRLHYSIDFTKKQGYGRIEHLKTLEQNVELAAAELKADEKSHAV
ILGDTRYGSEEKGTYHLALFGDRAQEIAGSATVKIGEKVHEIGIAGKQGSGGGGVAADIGAGLAD
ALTAPLDHKDKGLQSLTLDQSVSKNEKLKLAAQGAEKTYGNGDSLNTGKLKNDKVSREDFIRQIE
VDGQLITLESGEFQVYKQSHSALTAFQTEQIQDSEHSGKMVAKRQFRIGDIAGEHTSFDKLPEGG
RATYRGTAFGSDDAGGKLTYTIDFAAKQGNGKIEHLKSPELNVDLAAADIKPDGKRHAVISGSVL
YNQAEKGSYSLGIFGGKAQEVAGSAEVKTVNGIRHIGLAAKQ
>SEQ ID NO: 37 [fHbp fusion polypeptide, stabilised, fH binding disrupted,
& R41]
VAADIGAGLADALTAPLDHKDKSLQSLTLDQVVRKNEKLKLAAQGAEKTYGNGDSLNTGKLKNDK
VSRFDFIRQIEVDGQLITLESGEFQIYKQDHSAVVALQIEKINNPDKIDSLINQRSFRVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHAIGIAGKQGSGGGGVAADIGT
GLADALTAPLDHKDKGLKSLTLEDVIPQNGTLTLSAQGAEKTFKAGDKDNSLNTGKLKNDKISRF
DFVQKIEVDGQTITLASGEFQIYKQNHSAVVALQIEKINNPDKTDSLINQRSFRVSGLGGEHTAF
NQLPGGKAEYHGKAFSSDDPNGRLHYSIDFTKKQGYGRIEHLKTLEQNVELAAAELKADEKSHAV
ILGDTRYGSEEKGTYHLALFGDRAQEIAGSATVKIGEKVHAIGIAGKQGSGGGGVAADIGAGLAD
ALTAPLDHKDKGLQSLTLDQSVSKNEKLKLAAQGAEKTYGNGDSLNTGKLKNDKVSRFDFIRQIE
VDGQLITLESGEFQVYKQSHSALTAFQTEQIQDSEHSGKMVAKRQFRIGDIAGEHTSFDKLPEGG
RATYRGTAFGSDDAGGKLTYTIDFAAKQGNGKIEHLKSPELNVDLAAADIKPDGKRHAVISGSVL
YNQAEKGSYSLGIFGGKAQEVAGSAEVKTVNGIRHIGLAAKQ
>SEQ ID NO: 38 [fHbp fusion polypeptide, stabilised, fH binding disrupted,
& R41]
MGPDSDRLQQRRVAADIGAGLADALTAPLDHKDKSLQSLTLDQVVRKNEKLKLAAQGAEKTYGNG
DSLNTGKLKNDKVSRFDFIRQIEVDGQLITLESGEFQIYKQDHSAVVALQIEKINNPDKIDSLIN
QRSFRVSGLGGEHTAFNQLPDGKAEYHGKAFSSDDAGGKLTYTIDFAAKQGHGKIEHLKTPEQNV
ELAAAELKADEKSHAVILGDTRYGSEEKGTYHLALFGDRAQEIAGSATVKIGEKVHAIGIAGKQG
SGGGGVAADIGTGLADALTAPLDHKDKGLKSLTLEDVIPQNGTLTLSAQGAEKTFKAGDKDNSLN
TGKLKNDKISRFDFVQKIEVDGQTITLASGEFQIYKQNHSAVVALQIEKINNPDKTDSLINQRSF
RVSGLGGEHTAFNQLPGGKAEYHGKAFSSDDPNGRLHYSIDFTKKQGYGRIEHLKTLEQNVELAA
AELKADEKSHAVILGDTRYGSEEKGTYHLALFGDRAQEIAGSATVKIGEKVHAIGIAGKQGSGGG
GVAADIGAGLADALTAPLDHKDKGLQSLTLDQSVSKNEKLKLAAQGAEKTYGNGDSLNTGKLKND
KVSRFDFIRQIEVDGQLITLESGEFQVYKQSHSALTAFQTEQIQDSEHSGKMVAKRQFRIGDIAG
EHTSFDKLPEGGRATYRGTAFGSDDAGGKLTYTIDFAAKQGNGKIEHLKSPELNVDLAAADIKPD
GKRHAVISGSVLYNQAEKGSYSLGIFGGKAQEVAGSAEVKTVNGIRHIGLAAKQ
>SEQ ID NO: 39 [fHbp fusion polypeptide, stabilised]
Where X at residue 123 is any amino acid other than L and X at residue 379
is any amino acid other than L.
VAADIGAGLADALTAPLDHKDKSLQSLTLDQSVRKNEKLKLAAQGAEKTYGNGDSLNTGKLKNDK
VSRFDFIRQIEVDGQLITLESGEFQIYKQDHSAVVALQIEKINNPDKIDSLINQRSFXVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHEIGIAGKQGSGGGGVAADIGT
GLADALTAPLDHKDKGLKSLTLEDSIPQNGTLTLSAQGAEKTFKAGDKDNSLNTGKLKNDKISRF
DFVQKIEVDGQTITLASGEFQIYKQNHSAVVALQIEKINNPDKTDSLINQRSFXVSGLGGEHTAF
NQLPGGKAEYHGKAFSSDDPNGRLHYSIDFTKKQGYGRIEHLKTLEQNVELAAAELKADEKSHAV
ILGDTRYGSEEKGTYHLALFGDRAQEIAGSATVKIGEKVHEIGIAGKQGSGGGGVAADIGAGLAD
ALTAPLDHKDKGLQSLTLDQSVRKNEKLKLAAQGAEKTYGNGDSLNTGKLKNDKVSREDFIRQIE
VDGQLITLESGEFQVYKQSHSALTAFQTEQIQDSEHSGKMVAKRQFRIGDIAGEHTSFDKLPEGG
RATYRGTAFGSDDAGGKLTYTIDFAAKQGNGKIEHLKSPELNVDLAAADIKPDGKRHAVISGSVL
YNQAEKGSYSLGIFGGKAQEVAGSAEVKTVNGIRHIGLAAKQ
>SEQ ID NO: 40 [fHbp fusion polypeptide, stabilised]
VAADIGAGLADALTAPLDHKDKSLQSLTLDQSVRKNEKLKLAAQGAEKTYGNGDSLNTGKLKNDK
VSRFDFIRQIEVDGQLITLESGEFQIYKQDHSAVVALQIEKINNPDKIDSLINQRSFRVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHEIGIAGKQGSGGGGVAADIGT
GLADALTAPLDHKDKGLKSLTLEDSIPQNGTLTLSAQGAEKTFKAGDKDNSLNTGKLKNDKISRF
DFVQKIEVDGQTITLASGEFQIYKQNHSAVVALQIEKINNPDKTDSLINQRSFRVSGLGGEHTAF
NQLPGGKAEYHGKAFSSDDPNGRLHYSIDFTKKQGYGRIEHLKTLEQNVELAAAELKADEKSHAV
ILGDTRYGSEEKGTYHLALFGDRAQEIAGSATVKIGEKVHEIGIAGKQGSGGGGVAADIGAGLAD
ALTAPLDHKDKGLQSLTLDQSVRKNEKLKLAAQGAEKTYGNGDSLNTGKLKNDKVSRFDFIRQIE
VDGQLITLESGEFQVYKQSHSALTAFQTEQIQDSEHSGKMVAKRQFRIGDIAGEHTSFDKLPEGG
RATYRGTAFGSDDAGGKLTYTIDFAAKQGNGKIEHLKSPELNVDLAAADIKPDGKRHAVISGSVL
YNQAEKGSYSLGIFGGKAQEVAGSAEVKTVNGIRHIGLAAKQ
>SEQ ID NO: 41 [fHIbp fusion polypeptide, stabilised, R41S]
VAADIGAGLADALTAPLDHKDKSLQSLTLDQSVRKNEKLKLAAQGAEKTYGNGDSLNTGKLKNDK
VSRFDFIRQIEVDGQLITLESGEFQIYKQDHSAVVALQIEKINNPDKIDSLINQRSFRVSGLGGE
HTAFNQLPDGKAEYHGKAFSSDDAGGKLTYTIDFAAKQGHGKIEHLKTPEQNVELAAAELKADEK
SHAVILGDTRYGSEEKGTYHLALFGDRAQEIAGSATVKIGEKVHEIGIAGKQGSGGGGVAADIGT
GLADALTAPLDHKDKGLKSLTLEDSIPQNGTLTLSAQGAEKTFKAGDKDNSLNTGKLKNDKISRF
DFVQKIEVDGQTITLASGEFQIYKQNHSAVVALQIEKINNPDKTDSLINQRSFRVSGLGGEHTAF
NQLPGGKAEYHGKAFSSDDPNGRLHYSIDFTKKQGYGRIEHLKTLEQNVELAAAELKADEKSHAV
ILGDTRYGSEEKGTYHLALFGDRAQEIAGSATVKIGEKVHEIGIAGKQGSGGGGVAADIGAGLAD
ALTAPLDHKDKGLQSLTLDQSVSKNEKLKLAAQGAEKTYGNGDSLNTGKLKNDKVSREDFIRQIE
VDGQLITLESGEFQVYKQSHSALTAFQTEQIQDSEHSGKMVAKRQFRIGDIAGEHTSFDKLPEGG
RATYRGTAFGSDDAGGKLTYTIDFAAKQGNGKIEHLKSPELNVDLAAADIKPDGKRHAVISGSVL
YNQAEKGSYSLGIFGGKAQEVAGSAEVKTVNGIRHIGLAAKQ

1-15. (canceled)

16. An immunogenic composition comprising a fusion polypeptide, wherein the fusion polypeptide comprises all three of v1, v2 and v3 meningococcal Factor H binding protein (fHbp), wherein
(i) the v1 fHbp sequence comprises a sequence with at least 90% sequence identity to SEQ ID NO: 7;
(ii) the v2 fHbp sequence comprises a sequence with at least 80% sequence identity to SEQ ID NO: 8 and is modified to introduce a stabilizing substitution at position S32 that is an amino acid that is not serine and/or a stabilizing substitution at position L123 that is an amino acid that is not leucine;
(iii) the v3 fHbp sequence comprises a sequence with at least 80% sequence identity to SEQ ID NO: 9 and is modified to introduce a stabilizing substitution at position S32 that is an amino acid that is not serine and/or a stabilizing substitution at position L126 that is an amino acid that is not leucine; and
wherein the fHbp fusion polypeptide has an amino acid sequence of formula NH₂ -A-[-X-L]₃-B—COOH, wherein L is an optional linker amino acid sequence, A is an optional N terminal amino acid sequence, and B is an optional C terminal amino acid sequence and wherein X₁, X₂ and X₃ are the three meningococcal fHbp sequences in the order v2-v3-v1 from the N- to C-terminus, and
wherein the immunogenic composition is in combination with one or more saccharide antigen(s) from N. meningitidis serogroup A, C, W135 and/or Y.

17. The immunogenic composition according to claim 16, wherein the one or more saccharide antigen(s) from N. meningitidis serogroup A, C, W135 and/or Y is an oligosaccharide(s).

18. The immunogenic composition according to claim 17, wherein the oligosaccharide(s) are conjugated to one or more carrier protein(s).

19. The immunogenic composition according to claim 16, wherein the stabilizing substitution at position S32 of the v2 fHbp sequence is S32V and/or the stabilizing substitution at position L123 of the v2 fHbp sequence is L123R.

20. The immunogenic composition according to claim 16, wherein the stabilizing substitution at position S32 of the v3 fHbp sequence is S32V and/or the stabilizing substitution at position L126 of the v3 fHbp sequence is L126R.

21. The immunogenic composition according to claim 16, wherein the immunogenic composition is in combination with one or more of a purified meningococcal Neisserial Heparin Binding Antigen (NHBA) polypeptide; a purified meningococcal Neisserial Adhesion A (NadA) polypeptide and/or (iii) meningococcal outer membrane vesicles (OMVs) prepared from meningococcal strain NZ98/254.

22. The immunogenic composition according to claim 21, wherein the immunogenic composition is in combination with a purified meningococcal Neisserial Heparin Binding Antigen (NHBA) polypeptide, a purified meningococcal Neisserial Adhesion A (NadA) polypeptide, and (iii) meningococcal outer membrane vesicles (OMVs) prepared from meningococcal strain NZ98/254.

23. The immunogenic composition of claim 21, wherein the meningococcal NHBA polypeptide comprises an amino acid sequence having 90% or more identity to SEQ ID NO: 12.

24. The immunogenic composition according to claim 23, wherein the NHBA polypeptide is present at a concentration between 50 μg/ml and 150 μg/ml.

25. The immunogenic composition of claim 21, wherein the meningococcal NadA polypeptide comprises an amino acid sequence having 90% or more identity to SEQ ID NO: 17.

26. The immunogenic composition according to claim 25, wherein the NadA polypeptide is present at a concentration between 50 μg/ml and 150 μg/ml.

27. The immunogenic composition according to claim 16, wherein the fusion fHbp polypeptide is present at a concentration between 50 μg/ml and 150 μg/ml.

28. The immunogenic composition according to claim 21, wherein the immunogenic composition comprises the meningococcal OMVs at a concentration between 5 μg/ml and 30 μg/ml.

29. The immunogenic composition according to claim 28, wherein the meningococcal OMVs are at a concentration between 10 μg/ml and 15 μg/ml.

30. The immunogenic composition according to claim 16, wherein the immunogenic composition further comprises an adjuvant.

31. The immunogenic composition according to claim 30, wherein the adjuvant comprises an aluminum hydroxide adjuvant.

32. An immunogenic composition comprising meningococcal outer membrane vesicles in combination with one or more of (i) a NHBA polypeptide (ii) a NadA polypeptide and/or (iii) a fusion polypeptide comprising all three of v1, v2 and v3 meningococcal fHbp; wherein the outer membrane vesicles (OMVs) are present at a concentration between 5-30 g/ml.

33. An immunogenic composition comprising a fusion polypeptide, wherein the fusion polypeptide comprises all three of v1, v2 and v3 meningococcal Factor H binding protein (fHbp), in combination with one or more of: (i) a purified meningococcal Neisserial Heparin Binding Antigen (NHBA) polypeptide, (ii) a purified meningococcal Neisserial Adhesion A (NadA) polypeptide, and/or (iii) meningococcal outer membrane vesicles (OMVs).


SEQUENCE LISTING (44 sequences)

SEQ ID NO: 1 - protein, 274 aa, Neisseria meningitidis
MNRTAFCCLSLTTALILTACSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 3 - protein, 281 aa, Neisseria meningitidis
MNRTAFCCLSLTTALILTACSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 4 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 5 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 6 - protein, 262 aa, Neisseria meningitidis
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 7 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 8 - protein, 247 aa, Neisseria meningitidis
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 9 - protein, 250 aa, Neisseria meningitidis
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 10 - protein, 776 aa, synthetic construct
  REGION 1..776: note = source = /note="Description of Artificial Sequence: Synthetic polypeptide"
MGPDSDRLQQRRVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGPDSDRLQQRRVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 11 - protein, 488 aa, Neisseria meningitidis
MFKRSVIAMACIFALSACGGGGGGSPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 12 - protein, 464 aa, Neisseria meningitidis
SPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 13 - protein, 471 aa, Neisseria meningitidis
CGGGGGGSPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 14 - protein, 187 aa, Neisseria meningitidis
MKKIIFAALAAAAISTASAATYKVDEYHANARFAIDHFNTSTNVGGFYGLTGSVEFDQAKRDGKIDITIPIANLQSGSQHFTDHLKSADIFDAAQYPDIRFVSTKFNFNGKKLVSVDGNLTMHGKTAPVKLKAEKFNCYQSPMEKTEVCGGDFSTTIDRTKWGMDYLVNVGMTKSVRIDIQIEAAKQ

SEQ ID NO: 15 - protein, 168 aa, Neisseria meningitidis
ATYKVDEYHANARFAIDHFNTSTNVGGFYGLTGSVEFDQAKRDGKIDITIPIANLQSGSQHFTDHLKSADIFDAAQYPDIRFVSTKFNFNGKKLVSVDGNLTMHGKTAPVKLKAEKFNCYQSPMEKTEVCGGDFSTTIDRTKWGMDYLVNVGMTKSVRIDIQIEAAKQ

SEQ ID NO: 16 - protein, 644 aa, synthetic construct
  REGION 1..644: note = source = /note="Description of Artificial Sequence: Synthetic polypeptide"
MASPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQDGSGGGGATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMAKTEVCGGDFSTTIDRTKWGVDYLVNVGMTKSVRIDIQIEAAKQ

SEQ ID NO: 17 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 18 - protein, 341 aa, Neisseria meningitidis
LAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 19 - protein, 327 aa, Neisseria meningitidis
ATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

SEQ ID NO: 20 - protein, 202 aa, Neisseria meningitidis
MKPKPHTVRTLIAAIFSLALSGCVSAVIGSAAVGAKSAVDRRTTGAQTDDNVMALRIETTARSYLRQNNQTKGYTPQISVVGYNRHLLLLGQVATEGEKQFVGQIARSEQAAEGVYNYITVASLPRTAGDIAGDTWNTSKVRATLLGISPATQARVKIVTYGNVTYVMGILTPEEQAQITQKVSTTVGVQKVITLYQNYVQR

SEQ ID NO: 21 - protein, 178 aa, Neisseria meningitidis
SAVIGSAAVGAKSAVDRRTTGAQTDDNVMALRIETTARSYLRQNNQTKGYTPQISVVGYDRHLLLLGQVATEGEKQFVGQIARSEQAAEGVYNYITVASLPRTAGDIAGDTWNTSKVRATLLGISPATRARVKIVTYGNVTYVMGILTPEEQAQITQKVSTTVGVQKVITLYQNYVQR

SEQ ID NO: 22 - protein, 6 aa, synthetic construct
  REGION 1..6: note = source = /note="Description of Artificial Sequence: Synthetic peptide"
GSGGGG

SEQ ID NO: 23 - protein, 11 aa, synthetic construct
  REGION 1..11: note = source = /note="Description of Artificial Sequence: Synthetic peptide"
GPDSDRLQQRR

SEQ ID NO: 24 - protein, 13 aa, synthetic construct
  REGION 1..13: note = source = /note="Description of Artificial Sequence: Synthetic peptide"
GSGPDSDRLQQRR

SEQ ID NO: 25 - protein, 13 aa, synthetic construct
  REGION 1..13: note = source = /note="Description of Artificial Sequence: Synthetic peptide"
GKGPDSDRLQQRR

SEQ ID NO: 26 - protein, 12 aa, synthetic construct
  REGION 1..12: note = source = /note="Description of Artificial Sequence: Synthetic peptide"
MGPDSDRLQQRR

SEQ ID NO: 27 - skipped

SEQ ID NO: 28 - protein, 8 aa, synthetic construct
  REGION 1..8: note = source = /note="Description of Artificial Sequence: Synthetic peptide"
LEHHHHHH

SEQ ID NO: 29 - protein, 769 aa, synthetic construct
  REGION 1..769: note = source = /note="Description of Artificial Sequence: Synthetic polypeptide"
MGPDSDRLQQRRVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 30 - protein, 757 aa, synthetic construct
  REGION 1..757: note = source = /note="Description of Artificial Sequence: Synthetic polypeptide"
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 31 - protein, 757 aa, synthetic construct
  VAR_SEQ 240
  VAR_SEQ 496
  VAR_SEQ 543
  REGION 1..757: note = source = /note="Description of Artificial Sequence: Synthetic polypeptide"
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHXIGIAGKQGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHXIGIAGKQGSGGGGVAADIGAGLADALTAPLDHKDKGLQSLTLDQSVXKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 32 - protein, 757 aa, synthetic construct
  REGION 1..757: note = source = /note="Description of Artificial Sequence: Synthetic polypeptide"
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHAIGIAGKQGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 33 - protein, 757 aa, synthetic construct
  VAR_SEQ 32
  VAR_SEQ 123
  VAR_SEQ 285
  VAR_SEQ 379
  REGION 1..757: note = source = /note="Description of Artificial Sequence: Synthetic polypeptide"
VAADIGAGLADALTAPLDHKDKSLQSLTLDQXVRKNEKLKLAAQGAEKTYGNGDSLNTGKLKNDKVSRFDFIRQIEVDGQLITLESGEFQIYKQDHSAVVALQIEKINNPDKIDSLINQRSFXVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDXIPQNGTLTLSAQGAEKTFKAGDKDNSLNTGKLKNDKISRFDFVQKIEVDGQTITLASGEFQIYKQNHSAVVALQIEKINNPDKTDSLINQRSFXVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 34 - protein, 757 aa, synthetic construct
  REGION 1..757: note = source = /note="Description of Artificial Sequence: Synthetic polypeptide"
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 35 - protein, 757 aa, synthetic construct
  VAR_SEQ 32
  VAR_SEQ 123
  VAR_SEQ 285
  VAR_SEQ 379
  VAR_SEQ 543
  REGION 1..757: note = source = /note="Description of Artificial Sequence: Synthetic polypeptide"
VAADIGAGLADALTAPLDHKDKSLQSLTLDQXVRKNEKLKLAAQGAEKTYGNGDSLNTGKLKNDKVSRFDFIRQIEVDGQLITLESGEFQIYKQDHSAVVALQIEKINNPDKIDSLINQRSFXVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDXIPQNGTLTLSAQGAEKTFKAGDKDNSLNTGKLKNDKISRFDFVQKIEVDGQTITLASGEFQIYKQNHSAVVALQIEKINNPDKTDSLINQRSFXVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVXKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 36 - protein, 757 aa, synthetic construct
  REGION 1..757: note = source = /note="Description of Artificial Sequence: Synthetic polypeptide"
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 37 - protein, 757 aa, synthetic construct
  REGION 1..757: note = source = /note="Description of Artificial Sequence: Synthetic polypeptide"
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHAIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 38 - protein, 769 aa, synthetic construct
  REGION 1..769: note = source = /note="Description of Artificial Sequence: Synthetic polypeptide"
MGPDSDRLQQRRVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHAIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 39 - protein, 757 aa, synthetic construct
  VAR_SEQ 123: note = Any amino acid except Leu
  VAR_SEQ 379: note = Any amino acid except Leu
  REGION 1..757: note = source = /note="Description of Artificial Sequence: Synthetic polypeptide"
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFXVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFXVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 40 - protein, 757 aa, synthetic construct
  REGION 1..757: note = source = /note="Description of Artificial Sequence: Synthetic polypeptide"
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 41 - protein, 757 aa, synthetic construct
  REGION 1..757: note = source = /note="Description of Artificial Sequence: Synthetic polypeptide"
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 42 - protein, 10 aa, synthetic construct
  VARIANT 3..10: note = /replace=" "
  REGION 1..10: note = misc_feature - /note="Variant residues given in the sequence have no preference with respect to those in the annotations for variant positions"
  REGION 1..10: note = source = /note="Description of Artificial Sequence: Synthetic peptide"
GGGGGGGGGG

SEQ ID NO: 43 - protein, 10 aa, synthetic construct
  VARIANT 4..10: note = /replace=" "
  REGION 1..10: note = misc_feature - /note="Variant residues given in the sequence have no preference with respect to those in the annotations for variant positions"
  REGION 1..10: note = source = /note="Description of Artificial Sequence: Synthetic peptide"
HHHHHHHHHH

SEQ ID NO: 44 - protein, 10 aa, synthetic construct
  VARIANT 5..10: note = /replace=" "
  REGION 1..10: note = misc_feature - /note="Variant residues given in the sequence have no preference with respect to those in the annotations for variant positions"
  REGION 1..10: note = source = /note="Description of Artificial Sequence: Synthetic peptide"
HHHHHHHHHH